(19) United States
(12) Patent Application Publication
(10) Pub. No.: US 2022/0361871 A1
(43) Pub. Date: Nov. 17, 2022

(54) INTRACORPOREAL SUTURE TYING

(71) Applicant: Children's Medical Center Corporation, Boston, MA (US)

(72) Inventors: Heung Bae Kim, Weston, MA (US); Alexander Yang, Boston, MA (US); Kyle C. Wu, Boston, MA (US); Robert W. Crum, Brookline, MA (US); Farokh R. Demehri, Brookline, MA (US); Brianna L. Slatnick, Cambridge, MA (US)

(21) Appl. No.: 17/878,406

(22) Filed: Aug. 1, 2022

Related U.S. Application Data
(63) Continuation of application No. PCT/US21/16828, filed on Feb. 5, 2021.
(60) Provisional application No. 62/971,567, filed on Feb. 7, 2020.

(51) Int. Cl. A61B 17/062; A61B 17/04

(52) U.S. Cl. CPC A61B 17/0625; A61B 17/0483; A61B 17/0469

(57) ABSTRACT

Needle drivers and methods of the present disclosure are generally directed at intracorporeal suture tying using a winding technique. For example, a needle driver may include an elongate shaft having a magnet section between a proximal section and a distal section. A clamp coupled to the distal section may grasp a first end portion of a suture. A needle coupled to a second end portion of the suture may be magnetically secured to the magnet section, and the magnet section may then be rotated to form a loop in the suture. Forming a knot in the suture may include moving the loop over the first end portion of the suture grasped in the clamp. As compared to other intracorporeal suture tying techniques, intracorporeal knot tying carried out using the needle drivers and methods of the present disclosure may reduce the time and complexity associated with laparoscopic procedures.

### CROSS-REFERENCE TO RELATED APPLICATIONS

This application is a bypass continuation that claims priority to International Patent Application No. PCT/US21/16828 filed on Feb. 5, 2021, which claims priority to U.S. Provisional Patent App. No. 62/971,567 filed on Feb. 7, 2020, where the entire content of each of the foregoing is hereby incorporated by reference.

### TECHNICAL FIELD

The disclosure generally relates to intracorporeal suture tying and, more particularly, to a winding technique for intracorporeal suture tying.

### BACKGROUND

As compared to open surgery, minimal access surgical techniques can reduce postoperative recovery times and complications. Given these advantages, minimal access surgical techniques have become the standard approach for many common surgical procedures. In particular, laparoscopy is often preferred over open surgery for many types of surgical procedures.

Although minimal access offers advantages in laparoscopic procedures, it also imposes certain constraints. For example, the viability of a given technique for securing, ligating, or dividing tissue during laparoscopic surgery may depend on, among other things, the type of laparoscopic procedure being performed. Thus, while clips, staplers, and electrocautery may be useful in some cases, surgeons may nevertheless be required to carry out intracorporeal suturing and knot tying as part of certain laparoscopic procedures. However, when performed using laparoscopic instruments—particularly during procedures within narrow cavities—intracorporeal suture tying can be a slow and cumbersome part of a surgical procedure.

There remains a need for improved suture tying techniques suitable for use in minimally invasive surgical procedures.

### SUMMARY

Needle drivers and methods of the present disclosure are generally directed at intracorporeal suture tying using a winding technique. For example, a needle driver may include an elongate shaft having a magnet section between a proximal section and a distal section. A clamp coupled to the distal section may grasp a first end portion of a suture. A needle coupled to a second end portion of the suture may be magnetically secured to the magnet section, and the magnet section may then be rotated to form a loop in the suture. Forming a knot in the suture may include moving the loop over the first end portion of the suture grasped in the clamp. As compared to other intracorporeal suture tying techniques, intracorporeal knot tying carried out using the needle drivers and methods of the present disclosure may reduce the time and complexity associated with laparoscopic procedures.

In an aspect, a needle driver for a winding technique of intracorporeal suture tying disclosed herein includes an elongate shaft having a proximal section, a distal section, and a magnet section between the proximal section and the distal section. The needle driver may also include a handle assembly coupled to the proximal section of the elongate shaft, a clamp distal to the magnet section and coupled to the distal section of the elongate shaft, where the clamp is in mechanical communication with the handle assembly, and where the clamp is movable, via actuation of the handle assembly, between an open position and a closed position. The needle driver may also include an actuator coupled to the proximal section of the elongate shaft, the actuator activatable to rotate the magnet section of the elongate shaft relative to the clamp.

Implementations may include one or more of the following features. The actuator may include a trigger actuatable to rotate the magnet section of the elongate shaft relative to the clamp about a longitudinal axis defined by the elongate shaft. The actuator may include a knob, where predetermined rotation of the knob causes a corresponding rotation of the magnet section of the elongate shaft relative to the clamp about a longitudinal axis defined by the elongate shaft. The spacing between the magnet section and the clamp may remain constant, along a longitudinal axis defined by the elongate shaft, as the magnet section rotates about the longitudinal axis. The proximal section of the elongate shaft may rotate along with the magnet section of the elongate shaft as the magnet section rotates about a longitudinal axis defined by the elongate shaft. The distal section of the elongate shaft may be non-magnetic along at least an interface between the distal section and the magnet section. The magnet section may include one or more magnetized ferromagnetic materials. The magnet section may be rotatable at least 180 degrees in a first direction about a longitudinal axis defined by the elongate shaft. The magnet section may be rotatable at least 180 degrees in a second direction about the longitudinal axis, the second direction opposite the first direction. The magnet section may extend circumferentially about a longitudinal axis defined by the elongate shaft. The magnet section may include a plurality of magnets. An arrangement of at least some of the plurality of magnets may include one or more of (i) alternating dipole moments and (ii) a Halbach array. The clamp may be stationary as the magnet section of the elongate shaft rotates relative to the clamp about a longitudinal axis defined by the elongate shaft. The clamp may include a first jaw and a second jaw, the first jaw and the second jaw movable relative to one another via actuation of the handle assembly to move the clamp between the open position and the closed position. The clamp, the distal section of the elongate shaft, and the magnet section of the elongate shaft may be sized to be movable to a treatment site through a port having a diameter greater than about 3 mm and less than about 12 mm. The handle assembly and the actuator may be positioned relative to one another such that the handle assembly and the actuator are each independently actuatable by a user grasping the handle assembly using a neutral grip. The needle driver may further include a rod extending parallel to a longitudinal axis defined by the elongate shaft, where the clamp is coupled to the handle assembly via the rod, the handle assembly is actuatable to move the rod, and movement of the rod moves the clamp between the open position and the closed position. The needle driver may further include a shaft feature operable with the magnet section to secure a needle along the distal section of the elongate shaft in a predetermined manner. The shaft feature may define a channel structurally configured to receive at least a portion of the needle.

In an aspect, a method for intracorporeal suture tying disclosed herein includes grasping a first end portion of a suture in a clamp coupled to a distal section of an elongate shaft, the suture having a length extending through biological tissue, magnetically securing a needle to a magnet section of the elongate shaft, the magnet section proximal to the distal section of the elongate shaft, the needle coupled to a second end portion of the suture, with the needle magnetically secured to the magnet section, rotating the magnet section relative to the clamp to form the second end portion of the suture into a loop about the elongate shaft, and moving the needle and the loop in a distal direction, along the elongate shaft, such that the first end portion of the suture grasped in the clamp moves through the loop to form at least a portion of a knot in the length of the suture.

### BRIEF DESCRIPTION OF THE DRAWINGS

The foregoing and other objects, features, and advantages of the devices, systems, and methods described herein will be apparent from the following description of particular embodiments thereof, as shown in the accompanying figures. The figures are not necessarily to scale, emphasis instead being placed upon describing the principles of the devices, systems, and methods described herein. In the drawings, like reference numerals generally identify corresponding elements.

FIG. 1A is an isometric view of a needle driver.

FIG. 1B is a cross-sectional side view of the needle driver of FIG. 1A, the cross-section taken along 1B-1B in FIG. 1A.

FIG. 1C is a cross-sectional side view of the needle driver of FIG. 1A, the cross-sectional side view corresponding to the area of detail 1C in FIG. 1B.

FIG. 1D is a cross-sectional side view of the needle driver of FIG. 1A, the cross-sectional side view corresponding to the area of detail 1D in FIG. 1B.

FIG. 1E is a partially exploded, perspective view of the cross-section of the needle driver shown in FIG. 1D.

FIG. 2 is a schematic representation of the needle driver of FIG. 1 inserted into a subject via a laparoscopic port.

FIG. 3A is a first schematic representation of a method of intracorporeal suture tying.

FIG. 3B is a second schematic representation of a method of intracorporeal suture tying.

FIG. 3C is a third schematic representation of a method of intracorporeal suture tying.

FIG. 3D is a fourth schematic representation of a method of intracorporeal suture tying.

FIG. 3E is a fifth schematic representation of a method of intracorporeal suture tying.

FIG. 3F is a sixth schematic representation of a method of intracorporeal suture tying.

FIG. 4 is a perspective view of a magnet section of an elongate shaft of a needle driver.

FIG. 5 is a perspective view of a multidirectional follower of a needle driver.

FIG. 6 is a perspective view of a handle assembly including a toggle.

FIG. 7A is a perspective view of a handle assembly including two triggers.

FIG. 7B is a schematic representation of an initial position of a first pin and a second pin along a follower, with the first pin and the second pin independently movable relative to one another to rotate the follower in either of a clockwise direction or a counterclockwise direction.

FIG. 7C is a schematic representation of the first pin and the second pin along the follower of FIG. 7B, with the first pin and the second pin shown spaced from one another along the follower following a counterclockwise rotation of the follower.

FIG. 8A is a schematic representation of a flattened profile of the follower of FIG. 7B, depicting a temporal sequence of movements of the first pin and the second pin of FIG. 7B during counterclockwise rotation.

FIG. 8B is a schematic representation of a flattened profile of the follower of FIG. 7B, depicting a temporal sequence of movements of the first pin and the second pin of FIG. 7B during counterclockwise rotation.

FIG. 8C is a schematic representation of the flattened profile of the follower of FIG. 7B, depicting a temporal sequence of movements of the first pin and the second pin of FIG. 7B during clockwise rotation.

FIG. 8D is a schematic representation of the flattened profile of the follower of FIG. 7B, depicting a temporal sequence of movements of the first pin and the second pin of FIG. 7B during clockwise rotation.

FIG. 9 is a perspective view of a needle driver.

FIG. 10 is a close-up view of a magnet section and clamp of a needle driver.

FIG. 11 is a close-up view of a magnet section of a needle driver.

FIG. 12 is a partial cutaway view of a portion of the elongate shaft of the needle driver of FIG. 11.

### DETAILED DESCRIPTION

Embodiments will now be described more fully hereinafter with reference to the accompanying figures, in which certain embodiments are shown. The foregoing may, however, be embodied in many different forms and should not be construed as limited to the embodiments shown herein.

All documents mentioned herein are hereby incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, unless otherwise indicated or made clear from the context, the term “or” should generally be understood to mean “and/or,” and the term “and” should generally be understood to mean “and/or.”

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples or exemplary language (“e.g.,” “such as,” or the like) provided herein, is intended to better describe the embodiments, and does not pose a limitation on the scope of the embodiments or the claims. No language in the specification should be construed as indicating any unclaimed element as essential to the practice of the disclosed embodiments.

In the following description, it is understood that terms such as “first,” “second,” and the like, are words of convenience and are not to be construed as limiting terms, unless specifically stated.

As used herein, the term “clinician” shall be generally understood to refer to a care provider directly interacting or indirectly interacting (e.g., in instances of robotic surgery) with any portion of the devices and/or systems described herein in the course of preparing for or carrying out a medical procedure on a subject. Thus, for example, the term clinician is intended to include a physician, a nurse, or other medical professionals. Further, the term clinician may also or instead include support personnel assisting a medical professional in preparing for or carrying out a medical procedure.

Also, as used herein, the term “subject” shall be generally understood to be a mammal. Thus, the term subject shall be understood to include humans, as well as any other mammals treatable according to the techniques described herein. Stated differently, unless otherwise specified or made clear from the context, the devices, systems, and methods of the present disclosure shall be understood to be applicable to medical treatment of humans, veterinary treatment of other mammals, or teaching/research environments using mammalian tissue, such as tissue harvested from a cadaver.

Further, in the description that follows, the term “magnet section” shall be understood to include at least one magnetized material and, further or instead, the magnet section shall be understood to produce a magnetic field that may magnetically attract certain materials (e.g., metals such as alloys of iron, cobalt, nickel, and certain alloys of rare earth elements) to the magnet section. The magnetic field produced by the magnet section may be, for example, a persistent magnetic field such that the magnet section is a permanent magnet. However, in certain implementations, the magnet section may be an electromagnet such that the magnetic field may be varied or electrically activated.

Still further, in the description that follows needle drivers are described as being used to carry out intracorporeal knot tying. Thus, to the extent a needle is described herein as being magnetically coupled to a magnet section of a needle driver, it shall be understood that such a needle may be directly or indirectly coupled to an end portion of a suture unless otherwise specified or made clear from the context. Thus, for the sake of clarity in the description that follows, the position of only the needle is described and, unless otherwise specified or made clear from the context, it shall be understood that the position of the needle is a proxy for the position of the end portion of the suture coupled to the needle. Further, while a needle (attached to an end portion of the suture) is described as being magnetically coupled to a magnet section of the needle driver, it shall be appreciated that this is by way of an example offered for clear and efficient description, and any of various different magnetic materials may be coupled to the magnet section of the needle driver, as may be useful for any given use of the needle driver.

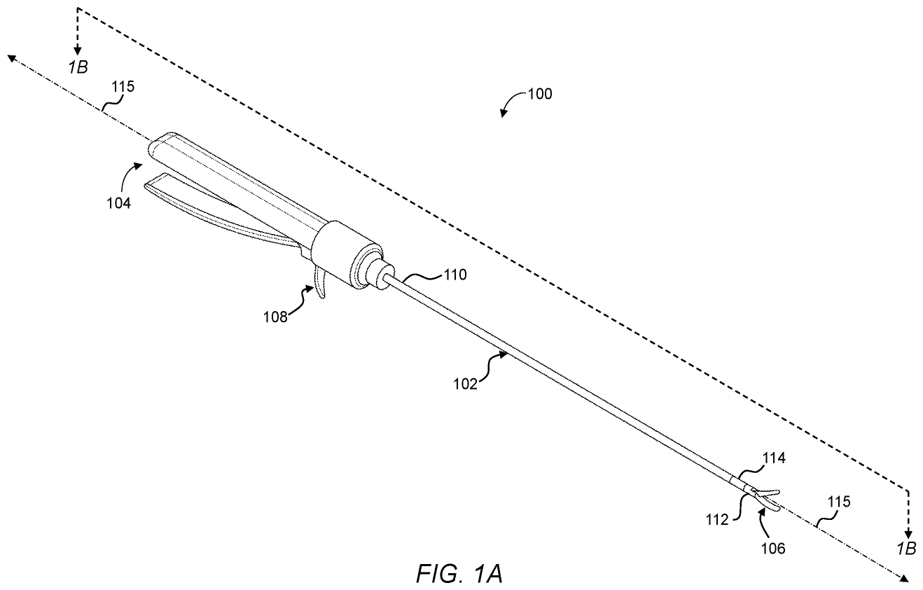

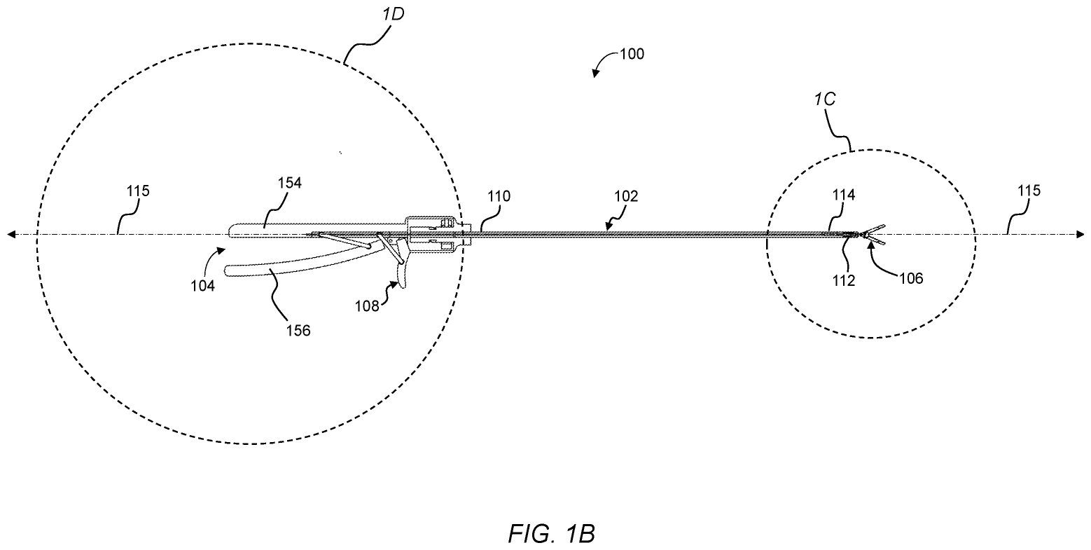

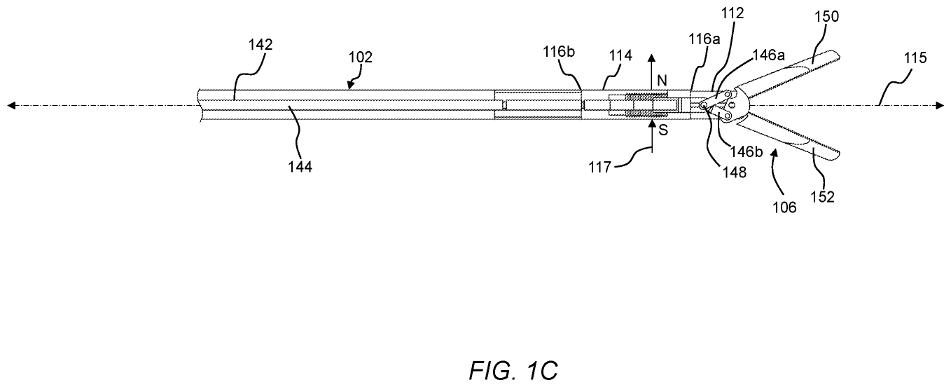

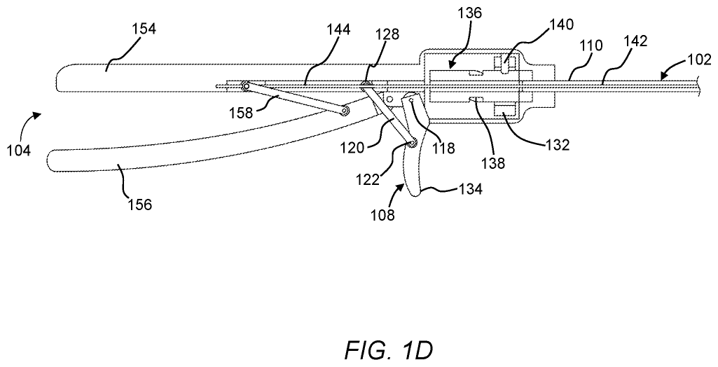

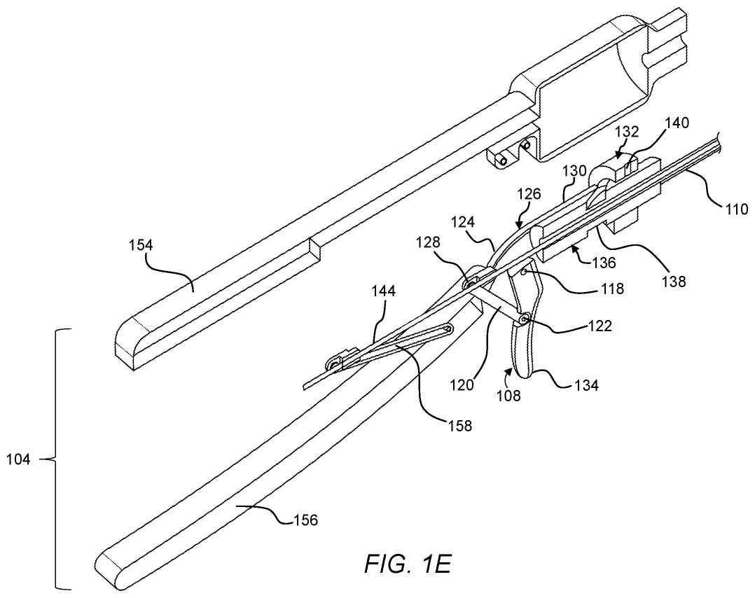

Referring now to FIGS. 1A-1E, a needle driver 100 may include an elongate shaft 102, a handle assembly 104, a clamp 106, and a trigger 108. The elongate shaft 102 may have a proximal section 110, a distal section 112, and a magnet section 114 therebetween. The handle assembly 104 and the trigger 108 may each be coupled to the proximal section 110 of the elongate shaft 102. At least a portion of the clamp 106 may be distal to the magnet section 114 and coupled to the distal section 112 of the elongate shaft 102. In general, the distal section 112 of the elongate shaft 102, the clamp 106, and the magnet section 114 may be sized for laparoscopic delivery to a treatment site of a subject while the handle assembly 104 and the trigger 108 are sized for direct or indirect manipulation by a clinician outside of the subject. More specifically, the handle assembly 104 may be actuatable to move the clamp 106 between an open position and a closed position, and the trigger 108 may be actuatable to rotate the magnet section 114 of the elongate shaft 102 relative to the clamp 106 (e.g., about a longitudinal axis 115 defined by the elongate shaft). Thus, the needle driver 100 may include an actuator in the form of a trigger 108 for actuating movement of the magnet section 114 of the needle driver 100. Through such separate control, the needle driver 100 may facilitate fine control over suture positioning and in doing so, may facilitate intracorporeal knot tying during laparoscopic procedures.

In use, as described in greater detail below, a clinician may magnetically couple a needle (with an attached suture) to the magnet section 114 and may actuate the handle assembly 104 to grasp another end portion of the suture with the clamp 106. In general, the needle shall be understood to be coupled to an end portion of a suture such that magnetic coupling of the needle to the magnet section 114 couples the end portion of the suture (that is attached to the needle) to the magnet section 114. As also described in greater detail below, the clinician may actuate the trigger 108 to rotate the magnet section 114 (and, thus, the needle magnetically coupled to the magnet section 114) such that rotation of the needle forms a loop in the end portion of the suture. As still further described in greater detail below, through relatively simple manipulation, the end portion of the suture grasped in the clamp 106 may be moved through this loop as part of a winding technique for intracorporeal suture tying. As compared to other intracorporeal suture tying techniques (e.g., the traditional loop technique), the winding technique for intracorporeal suture tying carried out using the needle driver 100 may advantageously reduce the time and complexity associated with laparoscopic procedures. Further, given the relative simplicity of suture manipulation in this winding technique, the use of the needle driver 100 may also reduce training time associated suture tying in a minimally invasive context and, more generally, may increase the viability of laparoscopic procedures in a variety of surgical environments.

In general, the elongate shaft 102 may be substantially rigid to resist deflection or other deformation in response to forces exerted on the elongate shaft 102 as the elongate shaft 102 is inserted into the subject and moved to a treatment site. Such rigidity may facilitate accurate positioning of the elongate shaft 102 at the treatment site using, for example, laparoscopic techniques. Further, or instead, the substantial rigidity of the elongate shaft 102 may be facilitate the winding technique for intracorporeal suture tying. For example, the elongate shaft 102 may be sufficiently rigid to maintain a constant spacing between the magnet section 114 and the clamp 106 along the longitudinal axis 115 during a procedure, as the magnet section 114 is actuated to rotate about the longitudinal axis 115. By limiting the magnet section 114 to a single degree of freedom for movement relative to the clamp 106 (specifically, rotation), the fixed axial spacing of the magnet section 114 relative to the clamp 106 may support fine control over positioning of the suture to carry out the winding technique for intracorporeal suture tying.

In certain implementations, the elongate shaft 102 may have a constant radial dimension from the proximal section 110 to the distal section 112. Thus, for example, the elongate shaft 102 may be a substantially right circular cylinder from the proximal section 110 to the distal section 112, although it shall be understood that other constant cross-sectional shapes may be used. For example, the elongate shaft 102 may be an elongate triangular prism or an elongate rectangular prism or another elongate polygon or the like. More specifically, in instances in which the elongate shaft 102 has a constant radial dimension from the proximal section 110 to the distal section 112, the magnet section 114 may be substantially flush with the proximal section 110 and the distal section 112 of the elongate shaft 102 to facilitate, for example, sliding a needle along the proximal section 110 or the distal section 112 into contact— and, thus, into magnetic coupling—with the magnet section 114. Further, or instead, the constant radial dimension of the elongate shaft 102 from the proximal section 110 to the distal section 112 may facilitate forming the elongate shaft 102 in a form factor resistant to buckling or other types of deformation while being small enough to be pass through a laparoscopic port.

In general, the elongate shaft 102 may be formed of any one or more of various different biocompatible materials suitable for imparting appropriate stiffness to the elongate shaft 102 and suitable for sterilization according to any one or more of various sterilization techniques known in the art. For example, the distal section 112 may be formed of a non-magnetic material (e.g., a hard plastic, a non-magnetized metal, or a combination thereof) along at least a first interface 116a between the distal section 112 and the magnet section 114. As may be appreciated, such a difference in magnetic properties at the first interface 116a may facilitate properly positioning a needle along the magnet section 114 by spatially directing magnetic forces to urge the needle in a predetermined orientation relative to the distal section 112. Further, or instead, such a difference in magnetic properties at the first interface 116a may facilitate rotating a needle by rotating the magnet section 114 relative to the distal section 112 according to the various different techniques described herein. In certain instances, the proximal section 110 may additionally, or alternatively, be formed of a non-magnetic material (e.g., a hard plastic, a non-magnetized material, or a combination thereof) along at least a second interface 116b, which may be useful for properly positioning a needle along the magnet section 114.

In certain implementations, the magnet section 114 may include one or more magnetized ferromagnetic materials. Examples of such magnetized ferromagnetic materials include, but are not limited to, one more of iron, cobalt, nickel, or alloys including any one or more of these materials, as well as alloys including certain rare earth elements such as neodymium. In certain implementations, the magnet section 114 may be formed entirely of one or more magnetized ferromagnetic materials, as may be useful for providing a strong magnet. In some instances, however, the magnet section 114 may be partially formed of one or more magnetized ferromagnetic materials dispersed (e.g., as flakes or other shapes) in a non-magnetized material (e.g., a polymer) to facilitate decoupling the strength of the magnetic field from the size and/or shape of the magnet section 114. Such decoupling may be useful, for example, in implementations in which a large surface area of the magnet section 114 is useful as a target for magnetically coupling a needle to the magnet section 114 while providing magnetic strength in a range suitable for decoupling the needle from the magnet section 114 using another laparoscopic instrument to move the needle relative to the magnet section 114, as described in greater detail below.

The magnet section 114 may be, for example, a permanent magnet having a persistent magnetic field in a fixed orientation relative to the magnet section 114 such that magnetically coupling a needle may be achieved by placing the needle in proximity to the magnet section 114. As a more specific example, the magnet section 114 may be a permanent magnet having a magnetic moment 117 substantially perpendicular to the longitudinal axis 115. In certain implementations, the orientation of the magnetic moment 117 substantially perpendicular to the longitudinal axis 115 may provide a particularly useful orientation of forces. That is, in instances in which the magnetic moment 117 is substantially perpendicular to the longitudinal axis 115, the resulting force distribution has a relatively large force in a direction substantially normal to the surface of the magnet section 114 and a relatively small force in a direction substantially parallel to the longitudinal axis 115 of the elongate shaft 102. Thus, while the relatively large normal force exerted by the magnet section 114 may hold the needle securely in place, the needle may nevertheless be removed from the magnet section 114 with relatively little force by moving the needle in a direction substantially parallel to the longitudinal axis 115 (e.g., in a proximal direction away from the clamp 106 or in a distal direction toward the clamp 106).

In certain implementations, the orientation of the magnetic moment 117 may be associated with a trade-off between the magnitude of the attractive force exerted by the magnetic moment 117 and the overall surface area of the magnet section 114 that may be attractive to the needle. In particular, with the magnetic moment 117 oriented substantially perpendicular to the longitudinal axis 115, about half of the circumference of the magnet section 114 may form a north pole (“N”) of a magnet while the remainder of the circumference of the magnet section 114 may form a south pole (“S”) of a magnet. That is, it shall be understood that orientation of the magnetic moment 117 substantially perpendicular to the longitudinal axis 115 may form two different faces along the magnet section 114—with one face attracting a magnetic material of a needle and the other face repelling the magnetic material of the needle. While orientation of the magnetic moment 117 substantially perpendicular to the longitudinal axis 115 may form a strong attractive force in a radial direction, this attractive force is along only about half of the circumference of the magnet section 114. As described in greater detail below, other orientations of the magnetic moment may be used to facilitate increasing a surface area useful for attracting the needle albeit, under otherwise identical conditions, at the cost of a lower attractive force. More generally, a variety of field-shaping techniques may be employed to shape the magnetic field in a manner that urges the needle into a predetermined orientation and/or retains the needle in the predetermined orientation relative to the longitudinal axis 115 of the elongate shaft 102.

As described in greater detail herein, mechanical fixtures may also or instead be used to mechanically urge a needle into the predetermined orientation relative to the elongate shaft 102.

In certain implementations, the magnet section 114 may extend circumferentially about the longitudinal axis 115 of the elongate shaft 102, as may be useful for facilitating placement of the needle. That is, with the magnet section 114 extending about the longitudinal axis 115 along the entire circumference of the elongate shaft 102, the needle may be placed anywhere along the magnet section 114 to magnetically couple the needle to the magnet section 114. This may be particularly useful in laparoscopic procedures characterized by a limited working volume that constrains positioning of laparoscopic instruments relative to one another. In some implementations, however, the magnet section 114 may extend only partially about the circumference of the elongate shaft 102, which may, for example, be useful for imposing a specific orientation of the needle with respect to the clamp 106 in instances in which such a specific orientation may be useful.

The magnet section 114 may be rotatable about the longitudinal axis 115 in any of various different directions or in any of various different degrees as may be suitable for a particular procedure. For example, to facilitate intracorporeal knot tying according to a winding technique described herein, the magnet section 114 may be rotatable, relative to the clamp 106, at least 180 degrees in a first direction (e.g., clockwise or counterclockwise) about the longitudinal axis 115 in response to each actuation of the trigger 108. That is, with each actuation of the trigger 108, the magnet section 114 may rotate about the longitudinal axis 115 by an amount sufficient to form a loop (or part of a loop) in a suture coupled to the needle magnetically coupled to the magnet section 114. In some implementations, the trigger 180 may rotate the magnetic section 114 relative to the clamp by about 180 degrees with each pull of the trigger 108 such that the clinician may pull the trigger 108 multiple times, as needed, to rotate the magnetic section 114 a total of about 360 degrees, about 540 degrees, about 720 degrees, and so on. Additionally, or alternatively, each pull of the trigger 108 may correspond to rotation of the magnetic section 114 by greater than about 180 degrees or, in some cases, greater than about 360 degrees. More generally, the exact amount of rotation associated with each pull of the trigger 108 may correspond to any one or more of various different amounts of rotation as may be useful to accommodate, among other things, the requirements of a given procedure, modifications to the winding technique of intracorporeal knot tying, clinician preference, and the like.

In certain implementations, rotation of the magnet section 114 relative to the clamp 106 may include rotation of the magnet section 114 relative to the distal section 112 of the elongate shaft 102. That is, as the magnet section 114 rotates relative to the clamp 106, the distal section 112 of the elongate shaft 102—and, thus, the clamp 106 coupled to the distal section 112 of the elongate shaft 102—may remain stationary such that an end portion of the suture grasped by the clamp 106 also remains stationary relative to the needle (and a portion of the suture coupled to the needle) rotating along with the magnet section 114. While the magnet section 114 may generally rotate relative to the distal section 112 of the elongate shaft 102, the proximal section 110 of the elongate shaft 102 may rotate along with the magnet section 114 as the magnet section 114 rotates about the longitudinal axis. Such rotation of the proximal section 110 of the elongate shaft 102 along with the magnet section 114 may be useful for transmitting force from the trigger 108 into rotational motion of the magnet section 114 using the proximal section 110 itself. For example, using the proximal section 110 to transmit force from the trigger 108 into rotational motion of the magnet section 114 may facilitate forming the elongate shaft 102 with a maximum cross-sectional dimension compatible with laparoscopic ports (e.g., greater than about 3 mm and less than about 12 mm). Further, or instead, using the proximal section 110 to transmit force from the trigger 108 into rotational motion of the magnet section 114 may facilitate robust force transmission while making use of few moving parts, as described in greater detail below.

In general, a force exerted on the trigger 108 may be translated into rotational motion of the magnet section 114 through any one or more of various different techniques suitable for the efficient transfer of force exerted by a clinician manipulating the needle driver 100 through single-handed operation. In this context, the efficient transfer of force shall be understood to include any of various different techniques in which a normal gripping force applied to the trigger 108 is translatable, subject to mechanical losses and without external force augmentation, into a rotational force at least sufficient to rotate the magnet section 114 about the longitudinal axis 115. Further, as used in this context, single-handed operation of the needle driver 100 shall be understood to refer to operation of the needle driver 100 by a clinician using only one hand, thus leaving the clinician's other hand free to manipulate another laparoscopic instrument to carry out any one or more of the various different intracorporeal knot tying techniques described herein.

As an example, the trigger 108 may form a portion of a four-bar linkage movable to translate actuation of the trigger 108 into a rotational force of the magnet section 114 about the longitudinal axis 115. The trigger 108 may be pivotably coupled to the handle assembly 104 at a first pivot 118 and the trigger 108 may be pivotably coupled to a trigger link 120 at a second pivot 122. In turn, the trigger link 120 may be pivotably coupled to a proximal end portion 124 of a coupler 126 at a third pivot 128. The coupler 126 may extend distally away from the third pivot 128 in a direction substantially parallel to the longitudinal axis 115 such that a distal end portion 130 of the coupler 126 is coupled to a slider 132 distal to the third pivot 128. In certain instances, the coupler 126 may be coupled to slider 132 at one or more portions of the slider 132 radially offset from the longitudinal axis 115, as may be useful in instances in which force transmission along the longitudinal axis 115 is associated with moving the clamp 106 between the open position and the closed position. More generally, however, it shall be appreciated that force transmission associated with the trigger 108 actuating the magnet section 114 and force transmission associated with actuating the clamp 106 may be offset from one another according to any one or more of various different orientations, as may be useful for achieving a form factor of the needle driver 100 suitable for single-handed operation by the clinician.

The trigger 108 may include a free portion 134 extending beyond the pivotable coupling of the trigger 108 and the trigger link 120 at the second pivot 122 and in a direction away from the pivotable coupling of the trigger 108 and the handle assembly 104. In use, the clinician may actuate the trigger 108 by pulling the free portion 134 of the trigger 108 in the proximal direction. As the free portion 134 of the trigger 108 moves in the proximal direction, the force on the trigger 108 may be transmitted to the coupler 126 via the trigger link 120 such that the coupler 126 moves in the proximal direction. Given the coupling between the coupler 126 and the pin 140, movement of the coupler 126 in the proximal direction, in turn, moves the pin 140 in the proximal direction. In particular, in the context of a four-bar linkage or similar arrangement of parts, the slider 132 may be a slider constrained to undergo only axial movement in a direction substantially parallel to the longitudinal axis 115. That is, the slider 132 may move in a direction substantially parallel to the longitudinal axis 115 as the slider 132 moves in the proximal direction during actuation and, further or instead, as the slider 132 moves in the distal direction to return to an original position.

In certain implementations, the needle driver 100 may include a follower 136 disposed in the handle assembly 104 and positioned (e.g., substantially centered) about the longitudinal axis 115. In general, the follower 136 may be coupled to the magnet section 114 such that rotation of the follower 136 may be translated into corresponding rotation of the magnet section 114. For example, in instances in which the proximal section 110 of the elongate shaft 102 is coupled to the magnet section 114, the follower 136 may be coupled to the proximal section 110 of the elongate shaft 102. Thus, continuing with this example, rotation of the follower 136 may be translated into rotation of the magnet section 114 via rotation of the proximal section 110 mechanically coupled between the follower 136 and the magnet section 114.

The follower 136 may define a recess 138, and the slider 132 may include a pin 140 engageable with the follower 136 along the recess 138 to rotate the follower 136. As an example, with the follower 136 disposed about the longitudinal axis 115, the recess 138 may extend along a curvilinear path extending both circumferentially about the longitudinal axis 115 and axially along the longitudinal axis 115 (e.g., a helical path). Additionally, or alternatively, the slider 132 (and, thus, the pin 140) may be restricted to move only substantially parallel to the longitudinal axis 115 while the follower 136 is movable only rotationally about the longitudinal axis 115. Continuing with this example, the pin 140 may be moved proximally into the recess 138 through initial actuation of the trigger 108 (e.g., via an axial force transmitted to the pin 140 via the coupler 126) and, as the pin 140 moves further in the proximal direction to engage the curvilinear path of the recess 138. As shall be appreciated, with the pin 140 constrained to move only substantially parallel to the longitudinal axis 115 and the follower 136 constrained to undergo only rotational motion about the longitudinal axis 115, movement of the pin 140 in the proximal direction, as the pin 140 is engaged with the recess 138, causes the follower 136 to rotate. As described above, rotation of the follower 136 may rotate the proximal section 110 and, thus, the magnet section 114 of the elongate shaft 102.

In general, it shall be understood that the direction and extent of rotation of the follower 136—and, thus, the extent and direction of the corresponding rotation of the magnet section 114—may be a function of the curvilinear shape of the recess 138. For example, depending on the shape of the recess 138, rotation of the follower 136 may be in a clockwise or counterclockwise direction as the pin 140 moves in a proximal direction and in the opposite direction as the pin 140 moves in the distal direction. Additionally, or alternatively, the degree of rotation of the follower 136 per actuation of the trigger 108 to move the pin 140 may be a function of a shape of the recess 138. Thus, for example, in instances in which it is desirable to rotate the magnet section 114 at least about 180 degrees in response to each actuation of the trigger 108, the shape of the recess 138 may extend circumferentially about the follower 136 by at least about 180 degrees. While the recess 138 may be shaped to rotate the magnet section 114 by at least about 180 degrees in some instances, it should be appreciated that the recess 138 may be any curvilinear shape suitable for achieving any amount(s) or direction(s) of rotation of the magnet section 114, as may be useful for addressing constraints of a given type of procedure, performing certain intracorporeal manipulations of a suture as part of a knot tying technique, or accommodating clinician preferences. Thus, for example, the recess 138 may be shaped to extend about the entire circumference of the follower 136 such that, through force exerted by the pin 140 moving along the recess 138, actuation of the trigger 108 may rotate the magnet section 114 by about 360 degrees, with less rotation or more rotation being achievable with an appropriately shaped recess 138. Further, in some instances, the recess 138 may be shaped such that the corresponding rotation of the magnet section 114 may be only in a single direction (e.g., clockwise or counterclockwise) such that actuation and return of the trigger 108 results only in rotation in the single direction, as may be useful for facilitating simplified operation of the needle driver 100. Alternatively, the recess 138 may be shaped such that the magnetic section 114 is biased to return to an original position following actuation of the trigger 108. That is, rotation of the magnet section 114 may be in a first direction (e.g., clockwise) as the trigger 108, and thus the pin 140, is pulled in the proximal direction, and rotation of the magnet section 114 may be in a second direction (e.g., counterclockwise), opposite the first direction, as the trigger 108, and thus the pin 140, moves in the distal direction to return to an original position. Such alternating rotation of the magnet section 114 resulting from actuation and return of the trigger 108 may, for example, facilitate complex manipulations of the suture or, in certain instances, to facilitate unwinding the suture to restart a knot tying process.

The elongate shaft 102 may define a lumen 142 extending from the proximal section 110 to the distal section 112. For example, in extending from the proximal section 110 to the distal section 112, the lumen 142 may extend through at least a portion of the magnet section 114 and, in certain instances, the magnet section 114 may define at least a portion of the lumen 142. In general, the lumen 142 may facilitate providing mechanical communication between one or more components of the handle assembly 104 and the clamp 106 without exposing such component(s) to tissue surrounding the elongate shaft 102 in use. That is, the elongate shaft 102 may encase one or more moving components extending through the lumen 142 such that the one or more moving component(s) do not pinch or otherwise inadvertently disturb tissue during a laparoscopic procedure. Additionally, or alternatively, the lumen 142 may be fluidically sealed, which may be useful for, among other things, initial sterilization and/or re-sterilization of the needle driver 100.

In certain implementations, the needle driver 100 may include a rod 144 extending parallel to the longitudinal axis 115. The clamp 106 may be directly or indirectly coupled to the handle assembly 104 via the rod 144. Through actuation of the handle assembly 104, the rod 144 may move in a proximal direction to actuate the clamp 106 from an open position to a closed position. Continuing with this example, as actuation of the handle assembly 104 is released, the rod 144 may move in a distal direction to release the clamp 106 from the closed position and return the clamp to the open position. That is, the clamp 106 may be normally open, which may be particularly useful for controlling grasping of an end of a suture as part of any one or more of the various different intracorporeal knot tying techniques described herein. While the handle assembly 104 and the clamp 106 may be coupled to one another with the clamp 106 in a normally open position, it shall be appreciated that the handle assembly 104 and the clamp 106 may alternatively be coupled to one another with the clamp 106 in a normally closed position.

In general, any one or more of various different force transmission mechanisms may be used to transmit force from the handle assembly 104 to the clamp 106 via the rod 144. As an example, the needle driver 100 may include a first leg 146a and a second leg 146b. The first leg 146a and the second leg 146b may each be pivotably attached to the rod 144 (e.g., along a portion of the rod 144 disposed in the lumen 142 along the distal section 112 of the elongate shaft 102) and to the clamp 106 such that axial movement of the rod 144 in the lumen 142 may pivot each of the first leg 146a and the second leg 146b. In turn, the pivoting movement of the first leg 146a and the second leg 146b in response to the axial movement of the rod 144 may move respective portions of the clamp 106 toward or away from the longitudinal axis 115 to open and close the clamp 106.

In certain instances, the first leg 146a and the second leg 146b may be pivotably attached to the rod 144 at a common pivot 148 to form a substantially “V” shape. The common pivot 148 may be disposed substantially along the longitudinal axis 115, which may be useful for achieving symmetric movement of portions of the clamp 106 relative to the longitudinal axis 115. Such symmetric movement of portions of the clamp 106 may be useful for achieving, for example, fine positional control of the clamp 106 which, in turn, may facilitate grasping a portion of the suture with the clamp 106 as part of any one or more of the various intracorporeal knot tying techniques described herein. Continuing with this example, as the rod 144 moves in the distal direction (e.g., through the release of actuation of the handle assembly 104), the first leg 146a and the second leg 146b may pivot about the common pivot 148 to move away from one another such that an included angle defined by the “V” shape increases. Movement of the first leg 146a and the second leg 146b away from one another, in turn, may be translated into movement of portions of the clamp 106 away from one another to move the clamp 106 from a closed position to an open position. Similarly, it shall be appreciated that movement of the rod 144 in the proximal direction (e.g., through actuation of the handle assembly 104) may move the first leg 146a and the second leg 146b about the common pivot 148 toward one another such that the included angle defined by the “V” shape decreases. Movement of the first leg 146a and the second leg 146b toward one another may be translated into movement of portions of the clamp 106 toward one another to move the clamp 106 from the open position to the closed position.

In general, the clamp 106 may include at least two members movable relative to one another to change the shape of the clamp 106 between the closed position and the open position. As used herein, the closed position shall be understood to correspond to any one of one or more shapes of the clamp 106 in which the at least two members are in contact with one another (or close enough relative to one another) to hold a portion of a suture with the clamp 106. Additionally, or alternatively, the open position of the clamp 106 shall be understood to correspond to any of one or more shapes of the clamp 106 in which the at least two members of the clamp 106 are positionable about the suture such that movement of the clamp 106 from the open position to the closed position holds the suture in place in the clamp 106. In general, the open position of the clamp 106 may include any one or more of various different shapes of the clamp 106 and, unless otherwise specified or made clear from the context, need not correspond to the most wide-open shape of the clamp 106.

In certain implementations, the clamp 106 may include a first jaw 150 and a second jaw 152 movable relative to one another via actuation of the handle assembly 104 to move the clamp 106 between the open position and the closed position. The first jaw 150 and the second jaw 152 may be any one or more of various different shapes that may be controlled through fine movements associated with grasping a suture without substantially compromising (e.g., inadvertently cutting) the suture. As an example, in the closed position of the clamp 106, contact between the first jaw 150 and the second jaw 152 may define a substantially planar contact area having a substantially uniform force distribution. Additionally, or alternatively, the first jaw 150 and the second jaw 152 may have substantially rounded distal portions to reduce the likelihood of inadvertently piercing or otherwise adversely impacting tissue at the treatment site. Further, or instead, the first jaw 150 and the second jaw 152 may contact one another at least along the longitudinal axis 115 such that grasping an end of the suture between the first jaw 150 and the second jaw 152 consistently locates the grasped end of the suture substantially in the vicinity of the longitudinal axis 115, where the grasped end of the suture can be advantageously positioned for carrying out any one or more of the various intracorporeal knot tying techniques described herein.

In general, the handle assembly 104 may include a first portion 154 and a second portion 156. The first portion 154 of the handle assembly 104 may house at least a portion of certain components of the needle driver 100, which may facilitate forming the needle driver 100 with a form factor suitable for single-handed operation by a clinician. As used in this context, the housing of components in the first portion 154 of the handle assembly 104 shall be understood to include any manner and form of supporting such components in at least a partially constrained orientation for proper transmission of forces according any one or more of the various different arrangements described herein. Additionally, or alternatively, the components housed in the first portion 154 of the handle assembly 104 may be at least partially separated from an environment outside of the first portion 154 of the handle assembly 104 to reduce the likelihood that grasping the handle assembly 104 may interfere with movement of components at least partially housed in the first portion 154 of the handle assembly 104. In certain instances, components housed in the first portion 154 of the handle assembly 104 may be fluidically sealed from an environment outside of the handle assembly 104, as may be useful for initially sterilizing and/or re-sterilizing the needle driver 100.

In certain instances, the first portion 154 of the handle assembly 104 may remain substantially stationary during actuation of the handle assembly 104, as may be useful for reliable and repeatable transmission of forces during operation of the needle driver 100. That is, the second portion 156 of the handle assembly 104 may be coupled to the rod 144 via a handle link 158. Continuing with this example, the second portion 156 of the handle assembly 104 may be movable toward the first portion 154 of the handle assembly 104. As the second portion 156 moves toward the first portion 154 of the handle assembly 104 in this way, the handle link 158 may move the rod 144 in the proximal direction which, in turn, may move the clamp 106 from the open position to the closed position (e.g., to grasp an end of the suture) or vice-versa. Additionally, or alternatively, the second portion 156 of the handle assembly 104 may be movable away from the first portion 154 of the handle assembly 104 to move the rod 144 in the distal direction which, via the handle link 158, may move the rod 144 in the distal direction to move the clamp 106 from the closed position to the open position (e.g., to release the end of the suture) or vice-versa.

The handle assembly 104 and the trigger 108 may be positioned relative to one another in any one or more of various different orientations suitable for single-handed operation of the needle driver 100 by the clinician. In this context, single-handed operation of the needle driver 100 by the clinician shall be understood to include operation of the handle assembly 104 and operation of the trigger 108 independently of one another using only one hand, without releasing the handle assembly 104 and/or without changing a grip of the one hand on the handle assembly 104 as the handle assembly 104 and the trigger 108 are variously actuated to carry out any one or more of the various intracorporeal knot tying techniques described herein. Thus, in certain instances, the handle assembly 104 and the trigger 108 may be positioned relative to one another such that the handle assembly 104 and the trigger 108 are each independently actuatable by a user (e.g., a clinician) grasping the handle assembly using a neutral grip. As an example, to facilitate single-handed operation of the needle driver 100 using a neutral grip, the handle assembly 104 and the trigger 108 may each be actuatable to move in a plane (such as the plane represented by cross-section 1B-1B in FIG. 1A) extending through the handle assembly 104. That is, actuation of the handle assembly 104 and the trigger 108 in a plane may provide a particularly useful distribution of forces that may reduce the likelihood of dropping or otherwise losing grip of the needle driver 100 as the handle assembly 104 and the trigger 108 are variously actuated.

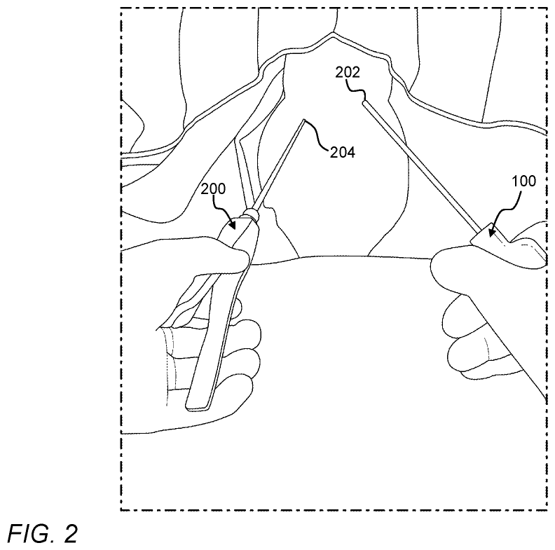

Referring now to FIG. 2, the needle driver 100 and forceps 200 may be insertable into a subject and movable to a treatment site through a first port 202 and a second port 204, respectively. In particular, at least the clamp 106, the distal section 112 of the elongate shaft 102, and the magnet section 114 of the elongate shaft 102 are insertable into the subject while the proximal section 110 of the elongate shaft 102 extends out of the subject such that the handle assembly 104 and the trigger 108 are outside of the subject, where the clinician may actuate the handle assembly 104 and the trigger 108. Given that it is generally desirable to use minimal port sizes to facilitate healing of the insertion site(s) and, thus, reduce the likelihood of post-operative infection, the components of the needle driver 100 that are inserted into the subject may be advantageously sized to be movable to a treatment site via the first port 202 having a size associated with typical laparoscopic procedures. Stated differently, the components of the needle driver 100 insertable into the subject may be sized such that use of the needle driver 100 does not generally require the use of larger port sizes. Thus, for example, the components of the needle driver 100 insertable into the subject (e.g., the clamp 106, the distal section 112, of the elongate shaft 102, and the magnet section 114 of the elongate shaft 102 in FIG. 1A) may be movable to a treatment site via through the first port 202 having a diameter greater than about 3 mm and less than about 12 mm.

The relative distance between the first port 202 and the second port 204 may be a function of accessibility of anatomy of the subject for the treatment being performed. Thus, in some instances, the relative distance between the first port 202 and the second port 204 may be wide. In other instances, the relative distance between the first port 202 and the second port 204 may be narrow. As compared to conventional intracorporeal knot tying techniques, the intracorporeal knot tying techniques described herein are relatively insensitive to the relative distance between the first port 202 and the second port 204.

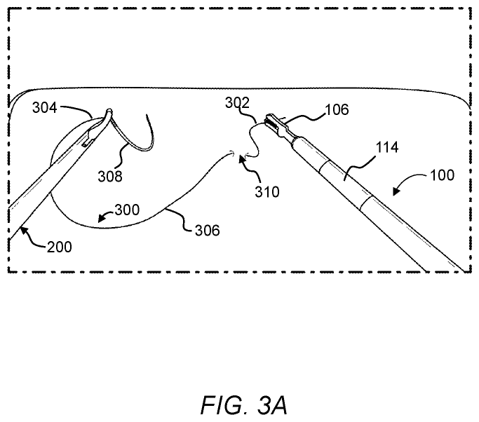

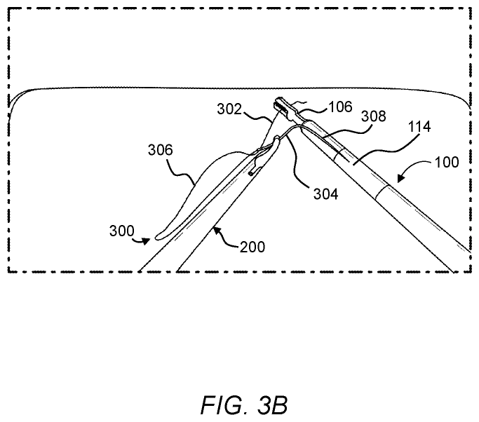

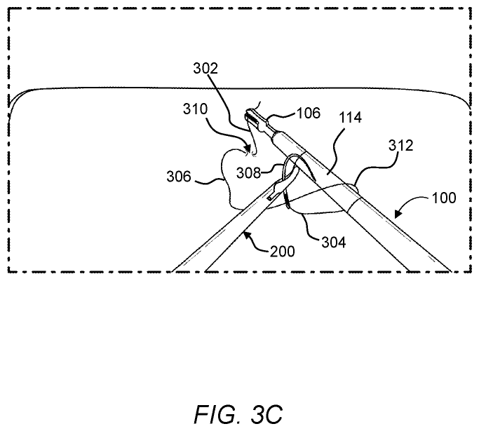

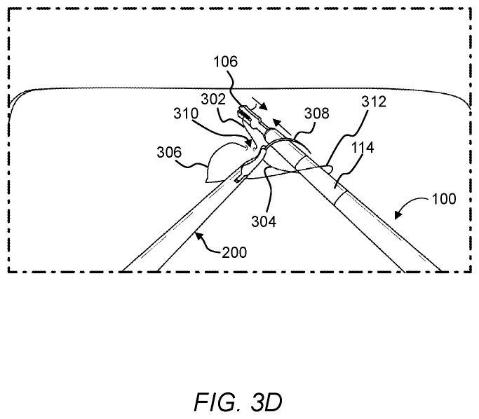

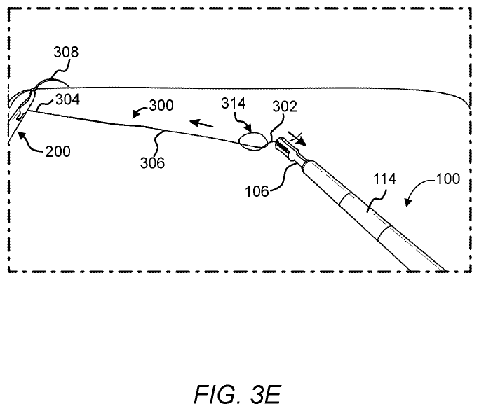

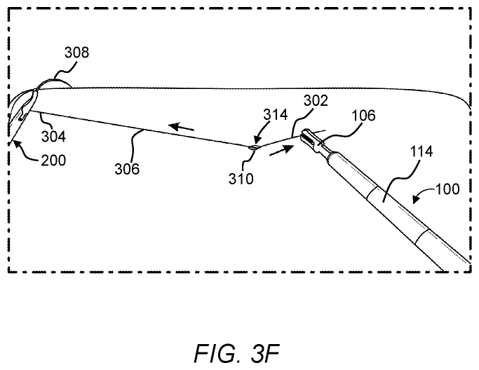

FIGS. 3A-3F are, collectively, a schematic representation of a method of intracorporeal suture tying. For the sake of clarity of explanation, the exemplary method is described with respect to the use of the needle driver 100 and the forceps 200 to carry out a winding technique for intracorporeal knot tying. It shall be understood that the exemplary method of intracorporeal knot tying may be carried out using any one or more of the various different aspects of the needle driver 100 described herein. Thus, unless otherwise specified or made clear from the context, a particular configuration of the needle driver 100 shall not necessarily be required to carry out the exemplary method of intracorporeal knot tying represented in FIGS. 3A-3F. Further, or instead, unless otherwise specified or made clear from the context, aspects of the exemplary method of intracorporeal knot tying represented in FIGS. 3A-3F may be carried out in any one or more of various different orders as may be suitable to carry out a winding technique for intracorporeal knot tying.

Referring now to FIG. 3A, a suture 300 may have a first end portion 302, a second end portion 304, and a length 306 extending therebetween. As this suggests, the term “length” in this context shall be understood to refer to a portion of the suture 300 between the first end portion 302 and the second end portion 304, and, unless otherwise specified or made clear from the context, this length is not necessarily intended to refer to a measured distance, nor is it intended to require a particular shape or orientation. In general, the second end portion 304 of the suture may be coupled (e.g., swaged) to a needle 308 movable through material 310 at the treatment site as may be may useful for securing the material 310 in the form of biological tissue and/or an implant, as the case may be, as part of a procedure. The needle 308 may be at least partially formed of any one or more of various different magnetic materials (e.g., ferromagnetic materials) suitable for magnetically coupling the needle 308 to the magnet section 114 with a force suitable for rotating needle 308 with the magnet section 114 to carry out a winding technique for laparoscopic suture tying.

The clamp 106 may grasp the first end portion 302 of the suture 300 extending through the material 310 at the treatment site. For example, the clinician may position the clamp 106 in the vicinity of the first end portion 302 and, with the clamp 106 so positioned, the clinician may actuate the clamp 106 to grasp the first end portion 302 of the suture 300. As used in this context, this grasping shall be generally understood to include restricting movement of the first end portion 302 of the suture 300 relative to the clamp 106. In instances in which the clamp 106 is substantially fixed in an axial and a radial direction relative to the magnet section 114, it shall be appreciated that grasping the first end portion 302 of the suture 300 may fix the first end portion 302 of the suture 300 relative to the magnet section 114, as is generally useful for forming a knot in the suture 300.

Referring now to FIGS. 3A and 3B, one or both of the second end portion 304 of the suture 300 or the needle 308 may be grasped in the forceps 200 to control movement of the second end portion 304 of the suture 300. In general, the forceps 200 and the clamp 106 may be moved relative to one another such that the first end portion 302 and the second end portion 304 of the suture 300 are, in turn, moved relative to one another. For example, through relative movement between the needle driver 100 and the forceps 200 (e.g., by moving the forceps 200 to the needle driver 100), the needle 308 may be moved into proximity with the magnet section 114 of the needle driver 100.

Referring now to FIG. 3B, with the needle 308 in proximity with the magnet section 114, the needle 308 may be magnetically secured to the magnet section 114. That is, proximity between the needle 308 and the magnet section 114 may include positioning the needle 308 in a magnetic field of the magnet section 114 such that magnetic force may draw the needle 308 into contact with the magnet section 114. Stated differently, the needle 308 may be magnetically secured to the magnet section 114 using only approximate positioning of the needle 308 relative to the magnet section 114. As compared to a technique requiring precise positioning, magnetically securing the needle 308 to the magnet section offers significant advantages with respect to the time and/or skill required to position the needle 308 relative to the magnet section 144 as part of a laparoscopic procedure.

Referring now to FIG. 3C, with the needle 308 magnetically secured to the magnet section 114, the needle driver 100 may be actuated to rotate the magnet section 114 relative to the clamp 106 to form the second end portion 304 of the suture 300 into a loop 312 about the elongate shaft 102 of the needle driver 100. That is, with the first end portion 302 of the suture 300 grasped in the clamp 106, the loop 312 may be formed proximal to the first end portion 302 of the suture 300 using only the relatively straightforward and familiar movement of drawing the trigger 108 (FIG. 1A) may facilitate achieving a complex aspect of intracorporeal knot tying with relatively little specialized skill.

In general, rotation of the magnet section 114 relative to the clamp 106 may facilitate robust and repeatable formation of the loop 312. For example, rotation of the magnet section 114 relative to the clamp 106 may facilitate keeping the first end portion 302 and the second end portion 304 of the suture 300 sufficiently spaced from one another as the second end portion 304 of the suture 300 is formed into the loop 312. In turn, such spacing may reduce the likelihood of unintended entanglement of the first end portion 302 with the second end portion 304 of the suture 300 as the second end portion 304 of the suture 300 is formed into the loop 312. In certain instances, rotation of the magnet section 114 relative to the clamp 106 may facilitate forming the loop 312 using less than a full rotation (e.g., greater than about 180 degrees of rotation and less than about 360 degrees of rotation) of the magnet section 114 which, in turn, may reduce the likelihood of unintended entanglement of the second end portion 304 with itself as the second end portion of the suture 300 is formed into the loop 312.

Referring now to FIG. 3E, the needle 308 and the loop 312 may be moved in a distal direction, along the elongate shaft 102, such that the first end portion 302 of the suture 300 grasped in the clamp 106 moves through the loop 312 to form at least a portion of a knot 314 in the length of the suture. For example, the forceps 200 may grasp the needle 308 and move the needle 308—and thus the loop 312—in the distal direction along the elongate shaft 102. Additionally, or alternatively, with the needle 308 grasped by the forceps 200, the needle driver 100 may be moved in a direction substantially opposite the distal direction of movement of the needle 308 and the loop 312 to facilitate moving the first end portion 302 of the suture 300 through the loop 312.

Referring now to FIG. 3F, with the first end portion 302 of the suture 300 passed through the loop 312, the needle driver 100 and the forceps 200 may be pulled in a direction substantially away from one another to tighten the loop 312 about the length 306 of the suture 300 such that the loop 312 is substantially fixed adjacent to the material 310 being secured at the treatment site. In general, unless otherwise specified or made clear from the context, it shall be understood that any one or more aspects of the technique described with respect to FIGS. 3A-3F may be repeated as necessary for intracorporeally tying a knot 314 at the treatment site.

While certain implementations have been described, other implementations are additionally or alternatively possible.

For example, while a needle driver has been described as including a handle assembly actuatable to actuate a clamp and a trigger actuatable to actuate a magnet section, it should be appreciated that movement of the trigger relative to the handle assembly may be advantageously limited in certain implementations. As an example, referring again to FIGS. 1A-1E, when the second portion 156 is positioned away from the first portion 154 of the handle assembly 104 such that the handle assembly 104 is not actuated and, thus, the clamp 106 is in an open position, the second portion 156 of the handle assembly 104 may abut the trigger link 120. Continuing with this example, the abutment of the second portion 156 of the handle assembly 104 against the trigger link 120 may prevent or limit actuation of the trigger 108 to rotate the magnet section 114. That is, in such implementations, the trigger 108 may be actuated only after the handle assembly 104 has been actuated, which may be useful for imposing an order to certain aspects associated with using needle driver 100 to carry out a winding technique for intracorporeal knot tying. In particular, in instances in which the trigger 108 is actuatable only after the handle assembly 104 has been actuated, the magnet section 114 may be rotated to form a loop in a suture only after the clamp 106 has been actuated to grasp another end of the suture. This may be useful, for example, to reduce the likelihood of inadvertent entanglement of both ends of the suture as the magnet section 114 is rotated to form a loop.

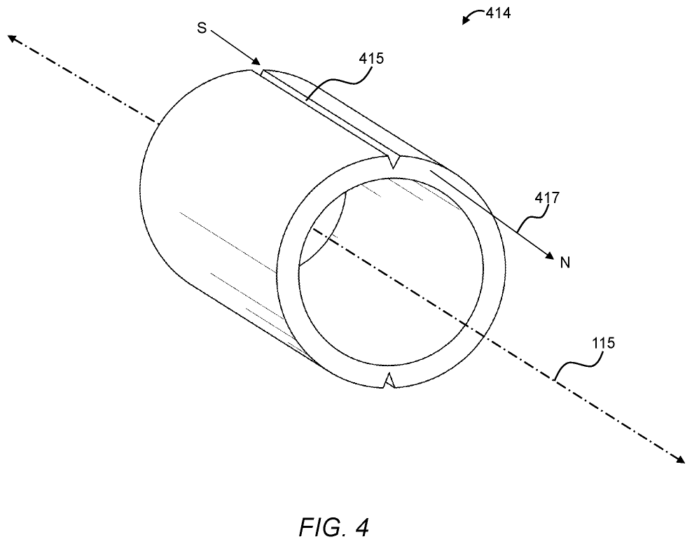

As another example, while a needle has been described as being magnetically securable to a magnet section of a needle driver, other techniques for securing the needle to the magnet section are additionally or alternatively possible. For example, referring now to FIG. 4, a magnet section 414 may define a channel 415, along which a needle may be positioned. Thus, the channel 415 may be structurally configured to receive, or otherwise engage with, at least a portion of a needle. Unless otherwise indicated, the magnet section 414 shall be understood to be similar to the magnet section 114 (FIG. 1A) and may be used interchangeably with the magnet section 114 (FIG. 1A) in the needle driver 100 (FIG. 1A) such that the magnet section 414 may rotate about the longitudinal axis 115. Accordingly, the magnet section 414 is not described separately, except to highlight differences or to emphasize certain aspects. Thus, for example, it shall be understood that, with a needle disposed in the channel 415, the magnet section 414 (and channel 415) may be rotatable in a manner analogous to rotation of the magnet section 114 (FIG. 1A) to form a loop in an end portion of a suture. That is, the channel 415 may include at least a portion substantially parallel to the longitudinal axis 115 to reduce the likelihood of inadvertent circumferential migration of the needle as the magnet section 414 rotates. Additionally, or alternatively, a plurality of instances of the channel 415 may be disposed circumferentially about the magnet section 414 to facilitate positioning the needle in a given instance of the channel 415. Further, or instead, while the channel 415 may include at least a portion substantially aligned with the longitudinal axis 115, it shall be appreciated that the channel 415 may have any one or more of various different orientations as may be useful for limiting potential migration in a given direction (e.g., axially, circumferentially, or a combination thereof).

As still another example, while a magnet section has been described as having a magnetic moment substantially perpendicular to a longitudinal axis defined by an elongate shaft at least partially formed by the magnet section, other orientations of the magnetic moment are additionally or alternatively possible. For example, the magnet section 414 may additionally, or alternatively, have a magnetic moment 417 substantially parallel to the longitudinal axis 115 such that a needle may be magnetically attracted to any point along the circumference of the magnet section 414. This may be useful, for example, for placing the needle along any portion of the magnet section 414. More generally, the orientation of the magnetic moment 417 may be oriented to facilitate achieving a predetermined alignment (e.g., perpendicular, parallel, or any orientation therebetween) of the needle relative to the longitudinal axis 115 (FIG. 1A) of the elongate shaft 102 (FIG. 1A). The predetermined alignment of the needle relative to the longitudinal axis 115 (FIG. 1A) may, in turn, repeatably and controllably orient the end portion of the suture swaged to the needle (e.g., the second end portion 304 swaged to the needle 308 shown in FIG. 3A). For example, the magnetic moment 417 may be oriented such that the needle is oriented relative to the longitudinal axis 115 (FIG. 1A) to position the swaged end portion of the suture to point generally in a proximal direction (toward the clinician) along the elongate shaft 102 to facilitate reaching through the loop to grasp the needle and/or the swaged end portion of the suture as part of any one or more of the intracorporeal knot tying procedures described herein (e.g., FIGS. 3A-3F).

As yet another example, while a needle driver has been described as rotating a magnet section in a single direction with a corresponding single trigger pull, it should be appreciated that other types of rotation of the magnet section are additionally, or alternatively, possible. For example, referring now to FIG. 5, a follower 536 may include a recess 538, which may include portions of substantially helical paths curving in opposite directions about the longitudinal axis 115. Unless otherwise indicated, the follower 536 shall be understood to be similar to the follower 136 (FIGS. 1D and 1E) in the needle driver 100 (FIG. 1A) such that any one or more of the magnet sections described herein (e.g., the magnet section 114 in FIG. 1A and/or the magnet section 414 in FIG. 4) may be rotated about the longitudinal axis 115 as the follower 536 is rotated about the longitudinal axis 115 under the force of the pin 140 (FIGS. 1D and 1E) moving in a direction substantially parallel to the longitudinal axis 115. Accordingly, the follower 536 is not described separately, except to highlight differences or to emphasize certain aspects.

In general, it shall be understood that the shape of the recess 538 may result in different rotational motion of a magnet section (e.g., the magnet section 114 in FIG. 1A and/or the magnet section 414 in FIG. 4, as the case may be) mechanically coupled to the follower 536 as compared to rotational motion of the same magnet section mechanically coupled to the follower 136. In particular, to the extent the recess 538 includes portions of substantially helical paths curving in opposite directions about the longitudinal axis 115, movement of the pin 140 (FIGS. 1D and 1E) along the recess 538 may rotate any one or more of the magnet sections described herein in two different rotational directions (namely, clockwise and counterclockwise), depending on where the pin 140 engages the recess 538 as the pin 140 moves along the recess 538. For example, in instances in which the needle driver 100 (FIG. 1A) includes the follower 536, any one or more of the magnet sections described herein may be rotatable at least about 180 degrees in a first direction about the longitudinal axis 115 and at least about 180 degrees in a second direction (opposite the first direction) about the longitudinal axis 115. In certain implementations, rotatability of the magnet section in two different directions may be useful for avoiding certain anatomical features. Further, or instead, rotatability of the magnet section by substantially the same amount in two different directions may be useful for consistently resetting the magnet section to a particular orientation following actuation.

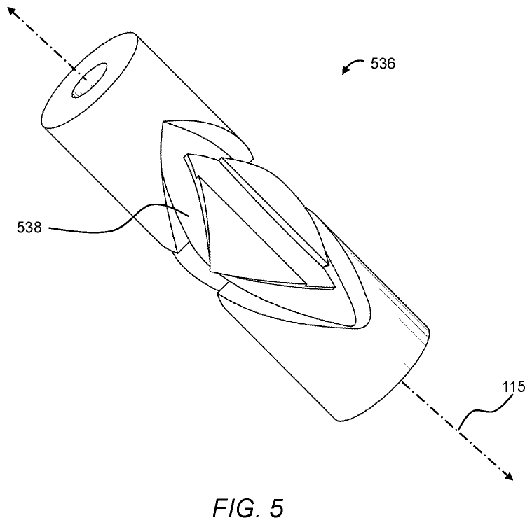

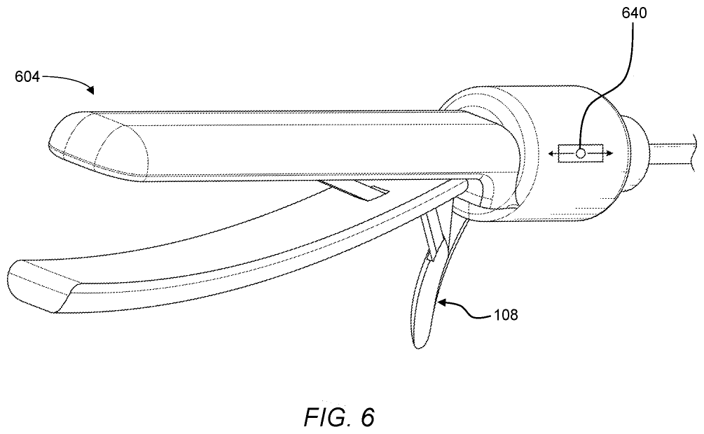

Referring now to FIGS. 5 and 6, a handle assembly 604 may include a toggle 640 movable by the clinician to change the direction of rotation of a magnetic section in response to a corresponding pull of a trigger. Unless otherwise indicated or made clear from the context, the handle assembly 604 shall be understood to be similar to the handle assembly 104 (FIG. 1A) such that any one or more of the magnet sections described herein (e.g., the magnet section 114 in FIG. 1A and/or the magnet section 414 in FIG. 4) may be rotated by actuation of the trigger 108. Accordingly, the handle assembly 604 is not described separately, except to highlight differences or to emphasize certain aspects.

In general, the toggle 640 may be mechanically coupled to the follower 536 or a pin (e.g., the pin 140 in FIG. 1D). In use, the toggle 640 may be actuatable by the clinician to change the position of the pin along the recess 538. In instances in which portions of the recess 538 correspond to oppositely curving helices, actuating the toggle 640 to change the relative position of the pin and the recess 538 may position the pin along a different helix defined by the recess 538. Continuing with this example, as the pin moves along a different helix defined by the recess 538, the direction of rotation of the follower 536 may change and, thus, the direction of the magnet section (e.g., the magnet section 114 in FIG. 1A and/or the magnet section 414 in FIG. 4) may change.

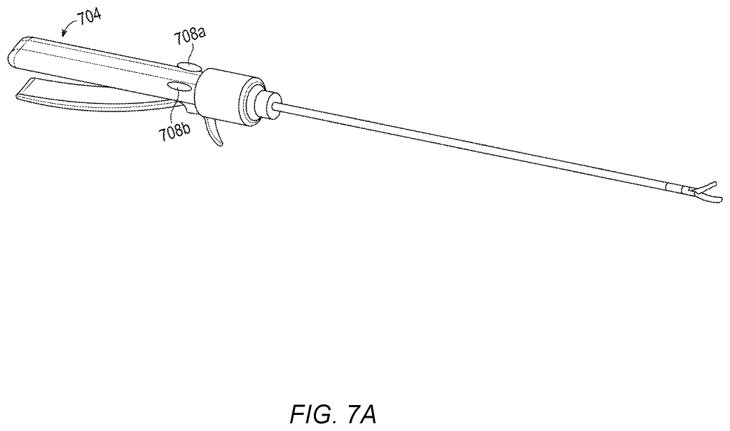

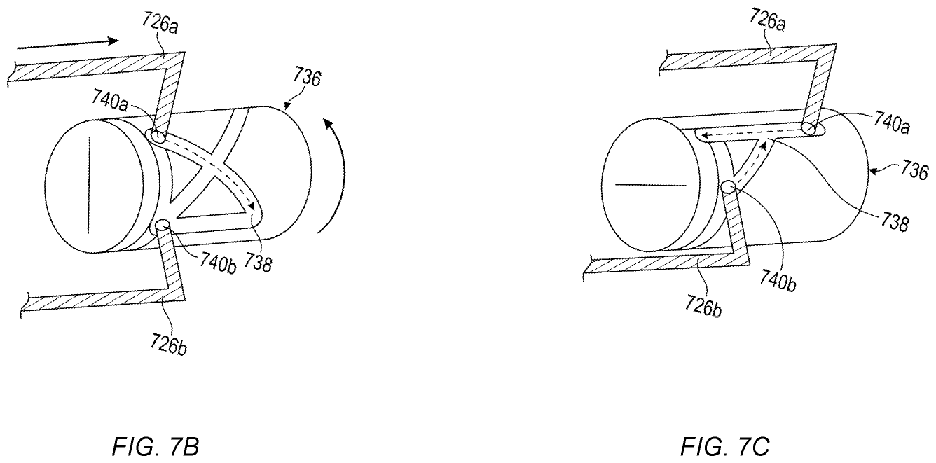

Further, or instead, while the rotational direction of the magnet section has been described as being adjustable based on actuation of a toggle, other approaches to changing direction of the magnet section are additionally or alternatively possible. For example, referring now to FIGS. 7A-7C, a handle assembly 704 may include a first trigger 708a, a second trigger 708b, a first coupler 726a, a second coupler 726b, a first pin 740a, a second pin 740b, and a follower 736. Unless otherwise indicated, elements designated with 700-series element numbers in FIGS. 7A-7C shall be understood to be similar to corresponding elements designated with 100-series elements, 400-series elements, 500-series elements, or 600-series elements in the other figures and, therefore, are not described separately, except to highlight differences or to emphasize certain aspects. Thus, for example, the handle assembly 704 shall be understood to be interchangeable with the handle assembly 104 (FIG. 1A) such that the handle assembly 704 is actuatable to open and close the clamp 106 (FIG. 1A). Additionally, or alternatively, the follower 736 may be coupled to any one or more of the various different magnet sections described herein (e.g., the magnet section 114 in FIG. 1C) via any one or more of the various different elongate shafts described herein (e.g., the elongate shaft 102 in FIG. 1B) such that, via the follower 736, actuation of the first trigger 708a and the second trigger 708b may rotate a corresponding magnet section in a clockwise direction or a counter-clockwise direction, as the case may be.

In general, the first trigger 708a may be coupled to the first pin 740a via the first coupler 726a, and the second trigger 708b may be coupled to the second pin 740b via the second coupler 726b. The first pin 740a and the second pin 740b may each be disposed in a recess 738 defined by the follower 736. The recess 738 may be shaped such that the first pin 740a and the second pin 740b may be actuated independently of one another and in any order to achieve any of various different combinations of rotation sequences of the follower 736. For example, the recess 738 may be shaped to allow the first trigger 708a to be actuated multiple times in a row to rotate the follower 736 through multiple, successive counterclockwise rotations. Further, or instead, the recess 738 may be shaped to allow the second trigger 708b to be actuated multiple times in a row to rotate the follower 736 through multiple, successive clockwise rotations. Still further or instead, the recess 738 may be shaped to allow the first trigger 708a and the second trigger 708b to be alternately actuated to achieve alternating clockwise and counterclockwise movement of the follower 736. In turn, any of the various different sequences of rotations of the follower 736 may be translated into a corresponding sequence of rotations of a magnet section (e.g., the magnet section 114 in FIG. 1C) coupled to the magnet section via an elongate shaft (e.g., the elongate shaft 102 in FIG. 1B).

In certain instances, actuation of the first trigger 708a may move the first pin 740a in a distal direction along the recess 738 defined by the follower 736 while the second pin 740b remains stationary such that the movement of the first pin 740a rotates the follower 736 in a counterclockwise direction. Such movement of the first pin 740a to rotate the follower 736 in a counterclockwise direction is depicted, for example, in FIGS. 7B and 7C. The recess 738 may be shaped such that, following this initial counterclockwise direction of the follower 736 (the state shown in FIG. 7C), either one of the first trigger 708a or the second trigger 708b may be actuated to rotate the follower 736 correspondingly in either direction.

For example, from the state shown in FIG. 7C, the first trigger 708a may reset (e.g., under the action of a return spring or the like) in a proximal direction such that the first pin 740a resets in the proximal direction. Through such resetting, the first trigger 708a may be actuated again to move the first pin 740a in the recess 738 to produce an additional counterclockwise rotation of the follower 736, and thus of a corresponding magnet section coupled to the follower 736. Additionally, or alternatively, following a reset of the first pin 740a from the state shown in FIG. 7C, the second trigger 708b may be actuated to move the second pin 740b in the recess 738 to produce a clockwise rotation of the follower 736, and thus of a corresponding magnet section coupled to the follower 736. Following such a clockwise rotation of the follower 736, the second pin 740b may reset (e.g., under the action of a return spring or the like) in a proximal direction such that the second trigger 708b may be actuated again to move the second pin 740b in the recess 738 to produce an additional clockwise rotation of the follower 736. It shall be appreciated that, for the sake of clear and efficient description, movement of the first pin 740a and the second pin 740b during counterclockwise rotation are shown by way of example in FIGS. 7B and 7C, and movement of the first pin 740a and the second pin 740b during clockwise rotation are not separately depicted.

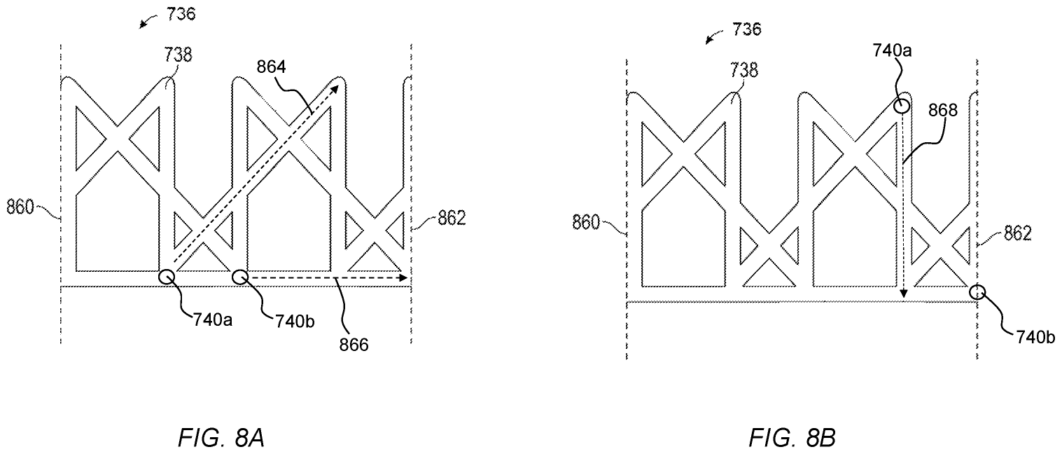

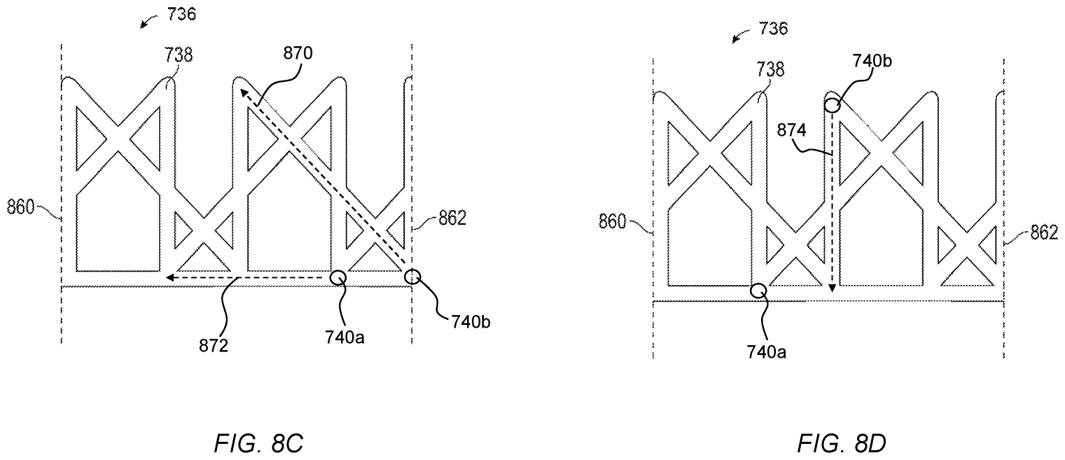

Referring now to FIGS. 8A-8D, the follower 736 and shape of the recess 738 are shown in a flattened profile in which a first end portion 860 and a second end portion 862 coincide with one another when the follower 736 is in the substantially cylindrical form shown in FIGS. 7B and 7C. Thus, while movement of the first pin 740a and the second pin 740b are described in terms of two-dimensional movements in the description that follows, it should be appreciated that this is for the sake of clarity of explanation and the actual movements of the first pin 740a and the second pin 740b are along the recess 738 defined along the substantially cylindrical surface of the follower 736. Thus, for example, diagonal movements depicted in the two-dimensional representations of FIGS. 8A-8D shall be understood to be along a substantially spiral paths along the follower 736.

Referring now to FIG. 8A, actuation of the first trigger 708a (FIG. 7A) may move the first pin 740a in a first direction 864 to rotate the follower 736 in the counterclockwise direction. As the follower 736 rotates in the counterclockwise direction, the second pin 740b may remain stationary such that a relative change of position of the second pin 740b and the follower 736 is realized in a second direction 866 as the follower 736 rotates in the counterclockwise direction. In three-dimensional space, it shall be appreciated that the second direction 866 is about at least a portion of the circumference of the follower 736.

Referring now to FIG. 8B, the first pin 740a may return to a reset position by moving in a third direction 868, which may correspond substantially to a proximal direction of the follower 736. With the first pin 740a and the second pin 740b reset following the movement shown in FIG. 8B, either trigger may be actuated to produce a corresponding clockwise or counterclockwise movement, as desired. For example, in instances in which additional counterclockwise movement of the follower 736 is desired for producing a corresponding additional counterclockwise movement of any one or more of the magnetic sections described herein, the sequence of movements depicted in FIGS. 8A and 8B may be repeated.

Referring now to FIG. 8C as another example, in instances in which clockwise movement of the follower 736 is desired, the second trigger 708b (FIG. 7A) may be actuated to move the second pin 740b in a fourth direction 870. As the follower 736 rotates in the clockwise direction, the first pin 740a may remain stationary such that a relative change of position of the first pin 740a and the follower 736 is realized in a fifth direction 872 as the follower 736 rotates in the clockwise direction.

Referring now to FIG. 8D, the second pin 740b may return to a reset position by moving in a sixth direction 874, which may correspond substantially to a proximal direction of the follower 736. With the first pin 740a and the second pin 740b reset following the movement shown in FIG. 8D, either trigger may be actuated to produce a corresponding clockwise or counterclockwise movement of the follower 736.

As yet another example, while a needle driver has been described as including a rod for actuation of a clamp and a portion of an elongate shaft for actuation of a magnet section, it should be appreciated that other actuation techniques are additionally or alternatively possible. For example, to the extent actuation of handle assemblies and triggers described herein have been described as being operable by pulling, one or more of the actuation members described herein may be implemented as a cable or another member operable using tension. Thus, for example, any one or more of the handle assemblies described herein may be coupled to a respective clamp via a cable that may be pulled to actuate the clamp. Additionally, or alternatively, any one or more of the handle assemblies described herein may be coupled to a respective magnet section via another cable that may be pulled to actuate the magnet section.

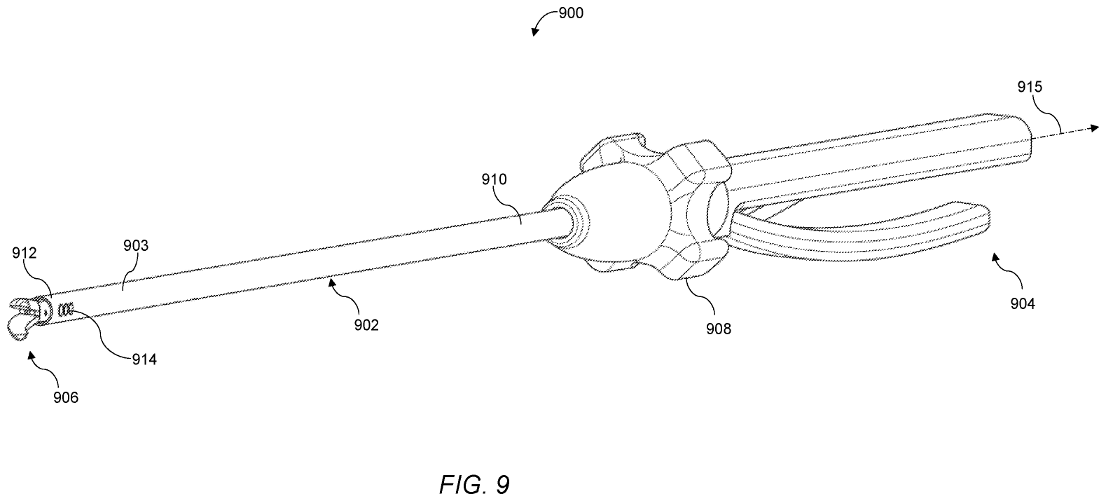

FIG. 9 is a perspective view of a needle driver. The needle driver 900 may be similar to those shown and described above and may generally include any of the features described above unless specifically noted otherwise. In general, the needle driver 900 may include an elongate shaft 902, a handle assembly 904, and a clamp 906. Further, the elongate shaft 902 may have a proximal section 910, a distal section 912, and a magnet section 914 therebetween. At least a portion of the clamp 906 may be distal to the magnet section 914 and coupled to the distal section 912 of the elongate shaft 902. In general, the distal section 912 of the elongate shaft 902, the clamp 906, and the magnet section 914 may be sized for laparoscopic delivery to a treatment site of a subject while the handle assembly 904 is sized for direct or indirect manipulation by a clinician outside of the subject. More specifically, the handle assembly 904 may be actuatable to move the clamp 906 between an open position and a closed position.

However, whereas the embodiment of FIGS. 1A-1E is described as having a trigger 108 as the actuator to manipulate (e.g., rotate or otherwise move) the magnet section 114 of the needle driver 100, in the embodiment shown by way of example in FIG. 9, a needle driver 900 may omit such a trigger mechanism and rotation may be controlled in a different manner. For example, the actuator for the needle driver 900 may include a knob 908 or the like to actuate and/or control movement of the magnet section 914 by converting rotational movement of the knob 908 into rotational movement of the magnet section 914.

To this end, the knob 908 may be engaged with at least a portion of the elongate shaft 902. For example, in certain embodiments, the knob 908 is coupled to an outer sleeve 903 of the elongate shaft 902 to which the magnet section 914 is also engaged (e.g., in a fixed manner), where the outer sleeve 903 is rotatable relative to the clamp 906 and/or other portions of the needle driver 900. In this manner, the knob 908 may be actuatable (e.g., by rotation thereof) to rotate the outer sleeve 903 of the elongate shaft 902, and thus rotate the magnet section 914 of the elongate shaft 902, relative to the clamp 906 (e.g., about a longitudinal axis 115 defined by the elongate shaft 902). Through separate control of the clamp 906 (via the handle assembly 904) and the magnet section 914 (via the knob 908), the needle driver 900 may facilitate fine control over suture positioning and in doing so, may facilitate intracorporeal knot tying during laparoscopic procedures as described herein.

The knob 908 may be sized and shaped for direct or indirect manipulation by a clinician outside of a subject during a laparoscopic procedure. In general, the knob 908 may define a region of the needle driver 900 that is engageable (e.g., via gripping)—directly or indirectly—by a clinician. Thus, the knob 908 may include one or more physical features (e.g., handholds, indentions, protrusions, and the like) sized and shaped to promote such engagement. Further, because the knob 908 may be structurally configured for rotating relative to one or more of the handle portion 904 and the clamp 906 of the needle driver 900, the knob 908 may include one or more features to assist a clinician in rotating the knob 908, and thus the magnet section 914, one or more discrete, predetermined motions. For example, the knob 908 may be engaged with one or more stops that limit or otherwise provide feedback regarding rotational movement of one or more of the knob 908 and the magnet section 914. This can include one or more features that provide tactile, audio, and/or visual feedback to a clinician, e.g., for discerning rotational movement in increments such as 90 degrees, 180 degrees, 360 degrees, or other amounts. The knob 908 may also or instead include one or more markings (or notable physical features) thereon that can provide feedback to a clinician regarding movement thereof and/or movement of the magnet section 914. It will be understood that rotation of the knob 908 may provide the same rotation of the magnet section 914 (i.e., 1:1), or rotation of the knob 908 may be mechanically coupled through a gearing system or the like to provide a different ratio of rotation for the magnet section 914 relative to the knob 908 (i.e., 2:1, 1:2, 3:1, 1:3, and so on).

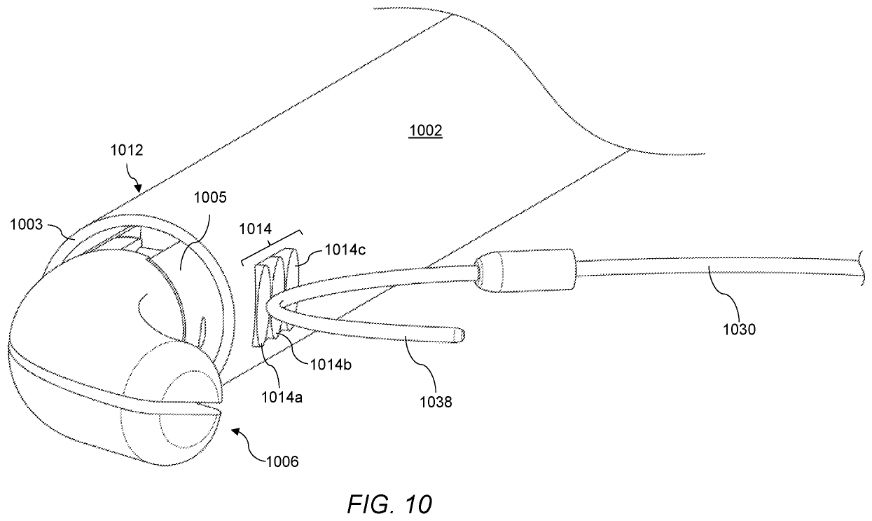

As also shown in FIG. 9, the needle driver 900 may include one or more magnets disposed along the magnet section 914. By way of example, three such magnets are shown in FIG. 9 and FIG. 10, which are described below.

FIG. 10 is a close-up view of a magnet section and clamp of a needle driver. The magnet section 1014 may include any of the magnet sections describe above. FIG. 10 thus shows the distal section 1012 of the elongate shaft 1002 of a needle driver as described herein, as well as a clamp 1006 disposed on an end of the needle driver. As explained above, the elongate shaft 1002 may include an outer sleeve 1003 that is movable (e.g., rotatable via rotation of a knob or the like) relative to the clamp 1006. In certain implementations, the clamp 1006 may be secured to an inner sleeve 1005 of the elongate shaft 1002 in a substantially fixed manner, where an outer sleeve 1003 to which the magnet section 1014 is coupled is rotatable relative to the inner sleeve 1005 and the clamp 1006. In this manner, rotation of the outer sleeve 1003 of the elongate shaft 1002 (via actuation of an actuator such as a knob, a trigger, or the like by a clinician) may provide rotation of the magnet section 1014 relative to the clamp 1006. Other mechanical configurations for moving the magnet section 1014 relative to the clamp 1006 may also or instead be used.

As shown in the figure, the magnet section 1014 may be structurally configured to engage with (e.g., via a magnetic force) a needle 1038 that is coupled (e.g., swaged) to a suture 1030. Thus, the needle 1038 may be at least partially formed of any one or more of the various different magnetic materials described herein and suitable for magnetically coupling the needle 1038 to the magnet section 1014 with a force suitable for rotating the needle 1038 with the magnet section 1014 to carry out a winding technique for laparoscopic suture tying.

The magnet section 1014 may include one or more magnets. By way of example and as shown in the figure, the magnet section 1014 may include at least three magnets—a first magnet 1014a, a second magnet 1014b, and a third magnet 1014c—although more or less magnets are possible. It will be understood that the number, configuration, alignment, and/or other properties (e.g., size, shape, materials, magnetic properties, and so on) of the magnets of the magnet section 1014 can be selected and specifically tailored for different use-cases and functionality, and the magnets in the magnet section 1014 may form a field-shaping array of magnets positioned to shape a magnetic field around the magnet section 1014 so that the magnet section 1014 urges the needle 1038 into a predetermined orientation relative to the elongate shaft 1002. For example, and as shown in the figure, the magnet section 1014 may include a plurality of magnets substantially aligned along a common axis (e.g., a longitudinal axis of the elongate shaft 1002), and having magnetic poles arranged to orient the needle 1038 in a specific manner to be more easily grasped by forceps or the like. Thus, in certain implementations, one or more magnets of the magnet section 1014 may be aligned along the longitudinal axis of the elongate shaft 1002.

More generally, the magnets of the magnet section 1014 can be arranged in a variety of manners to improve usability of the needle driver 900. By way of example, if multiple magnets of the magnet section 1014 are arranged linearly as shown in FIG. 10, they can be configured to have alternating dipole moments, which can create a stronger magnetic field along the length of the arrangement as opposed to multiple magnets that are oriented in the same direction. As another example, the magnets 1014 may be arranged in a Halbach array to create a stronger magnetic field on one side (e.g., toward an outer surface of the elongate shaft 1002) and a weaker magnetic field on the other side (e.g., toward an interior of the elongate shaft 1002). Other arrangements to manipulate and configure the magnetic fields for desired effects, e.g., to shape the magnetic field to be strongest in a line parallel to an axis of the elongate shaft 1002 so that the needle 1038 is urged into a similar alignment when contacting the magnet section 1014, are also known in the art and may usefully be employed to shape the magnetic field of the magnet section 1014 as described herein.

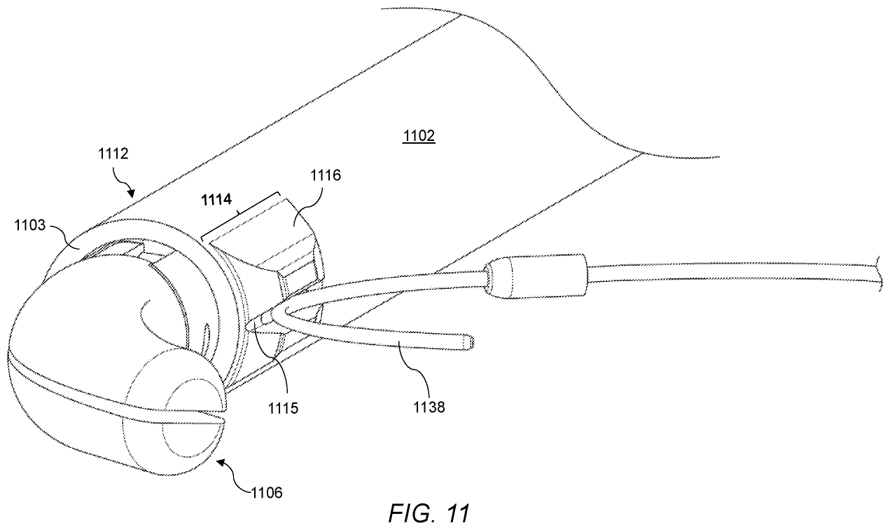

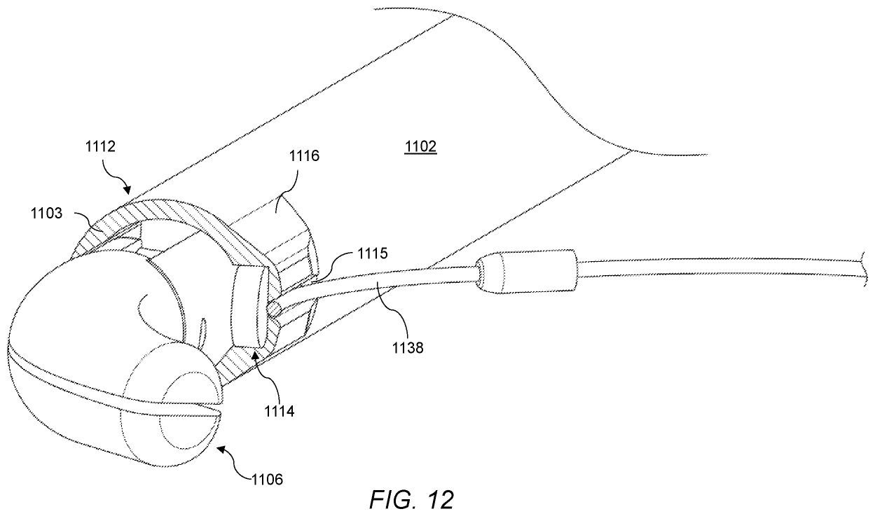

FIG. 11 is a close-up view of a magnet section of a needle driver, and FIG. 12 is a partial cutaway view of a portion of the elongate shaft of the needle driver of FIG. 11. As shown in these figures, the distal section 1112 of the elongate shaft 1102 of a needle driver may include a shaft feature 1116 that is structurally configured to mechanically securing a needle 1138 to the distal section 1112 in a predetermined orientation to carry out a winding technique for laparoscopic suture tying as described herein.

The shaft feature 1116 may be disposed on the elongate shaft 1102, and more particularly, the shaft feature 1116 may be disposed on an outer sleeve 1003 or other outer portion of the distal section 1112 of the elongate shaft 1102. The shaft feature 1116 may be structurally configured to retain the needle 1138 along the distal section 1112—e.g., in a predetermined position and/or orientation relative to the elongate shaft 1102. For example, the shaft feature 1116 may define a channel 1115 along which the needle 1138 may be positioned so that the needle 1138 is mechanically secured within the channel 1115 relative to the elongate shaft 1102. More generally, the shaft feature 1116 may define a window, a groove, an indentation, or other structure shaped to engage the needle 1138 with the assistance of magnetic forces in a desired position/orientation relative to the outer sleeve 1003 of the elongate shaft 1102. The shaft feature 1116 may generally be disposed at or near (e.g., adjacent to) the magnet section 1114, and may be movable with the magnet section 1114 (e.g., rotatable). In another aspect, the needle driver may provide a mechanism for moving the shaft feature 1116 relative to the magnet section 1114 in a manner that disengages the needle 1138 from the magnetic forces of the magnet section 1114. In another aspect, the shaft feature 1116 may include one or more magnets such that the shaft feature 1116 itself defines some or all of the magnet section 1114.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the devices, systems, and methods described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A needle driver for a winding technique of intracorporeal suture tying, the needle driver comprising:
an elongate shaft having a proximal section, a distal section;
a handle assembly coupled to the proximal section of the elongate shaft;
a clamp at the distal section of the elongate shaft, the clamp in mechanical communication with the handle assembly and the clamp including a first jaw and a second jaw configured to grasp a suture by moving relative to one another via actuation of the handle assembly to open and close the clamp;
a magnet section coupled to the elongate shaft between the proximal section and the distal section, the magnet section including a number of magnets creating a magnetic field shaped to orient a ferrous needle coupled to the suture in a predetermined alignment relative to the elongate shaft; and
an actuator coupled to the proximal section of the elongate shaft, the actuator activatable to rotate the elongate shaft to rotate the magnet section about an axis of the elongate shaft relative to the clamp while the clamp is closed about the suture.

2. A needle driver for a winding technique of intracorporeal suture tying, the needle driver comprising:
an elongate shaft having a proximal section, a distal section, and a magnet section between the proximal section and the distal section;
a handle assembly coupled to the proximal section of the elongate shaft;
a clamp distal to the magnet section and coupled to the distal section of the elongate shaft, the clamp in mechanical communication with the handle assembly, and the clamp movable, via actuation of the handle assembly, between an open position and a closed position; and
an actuator coupled to the proximal section of the elongate shaft, the actuator activatable to rotate the magnet section of the elongate shaft relative to the clamp.

3. The needle driver of claim 2, wherein the actuator includes a trigger actuatable to rotate the magnet section of the elongate shaft relative to the clamp about a longitudinal axis defined by the elongate shaft.

4. The needle driver of claim 2, wherein the actuator includes a knob, and wherein predetermined rotation of the knob causes a corresponding rotation of the magnet section of the elongate shaft relative to the clamp about a longitudinal axis defined by the elongate shaft.

5. The needle driver of claim 2, wherein spacing between the magnet section and the clamp remains constant, along a longitudinal axis defined by the elongate shaft, as the magnet section rotates about the longitudinal axis.

6. The needle driver of claim 2, wherein the proximal section of the elongate shaft rotates along with the magnet section of the elongate shaft as the magnet section rotates about a longitudinal axis defined by the elongate shaft.

7. The needle driver of claim 2, wherein the distal section of the elongate shaft is non-magnetic along at least an interface between the distal section and the magnet section.

8. The needle driver of claim 2, wherein the magnet section includes one or more magnetized ferromagnetic materials.

9. The needle driver of claim 2, wherein the magnet section is rotatable at least 180 degrees in a first direction about a longitudinal axis defined by the elongate shaft.

10. The needle driver of claim 9, wherein the magnet section is rotatable at least 180 degrees in a second direction about the longitudinal axis, the second direction opposite the first direction.

11. The needle driver of claim 2, wherein the magnet section extends circumferentially about a longitudinal axis defined by the elongate shaft.

12. The needle driver of claim 2, wherein the magnet section includes a plurality of magnets.

13. The needle driver of claim 12, wherein an arrangement of at least some of the plurality of magnets includes one or more of (i) alternating dipole moments and (ii) a Halbach array.

14. The needle driver of claim 2, wherein the clamp is stationary as the magnet section of the elongate shaft rotates relative to the clamp about a longitudinal axis defined by the elongate shaft.

15. The needle driver of claim 2, wherein the clamp includes a first jaw and a second jaw, the first jaw and the second jaw movable relative to one another via actuation of the handle assembly to move the clamp between the open position and the closed position.

16. The needle driver of claim 2, wherein the clamp, the distal section of the elongate shaft, and the magnet section of the elongate shaft are sized to be movable to a treatment site through a port having a diameter greater than about 3 mm and less than about 12 mm.

17. The needle driver of claim 2, wherein the handle assembly and the actuator are positioned relative to one another such that the handle assembly and the actuator are each independently actuatable by a user grasping the handle assembly using a neutral grip.

18. The needle driver of claim 2, further comprising a rod extending parallel to a longitudinal axis defined by the elongate shaft, wherein the clamp is coupled to the handle assembly via the rod, the handle assembly is actuatable to move the rod, and movement of the rod moves the clamp between the open position and the closed position.

19. The needle driver of claim 2, further comprising a shaft feature operable with the magnet section to secure a needle along the distal section of the elongate shaft in a predetermined manner.

20. The needle driver of claim 19, wherein the shaft feature defines a channel structurally configured to receive at least a portion of the needle.

21. A method for intracorporeal suture tying, the method comprising:
grasping a first end portion of a suture in a clamp coupled to a distal section of an elongate shaft, the suture having a length extending through biological tissue;
magnetically securing a needle to a magnet section of the elongate shaft, the magnet section proximal to the distal section of the elongate shaft, the needle coupled to a second end portion of the suture;
with the needle magnetically secured to the magnet section, rotating the magnet section relative to the clamp to form the second end portion of the suture into a loop about the elongate shaft; and
moving the needle and the loop in a distal direction, along the elongate shaft, such that the first end portion of the suture grasped in the clamp moves through the loop to form at least a portion of a knot in the length of the suture.
